(19) United States
(12) Patent Application Publication
(10) Pub. No.: US 2025/0018043 A1
(43) Pub. Date: Jan. 16, 2025

(54) COMPOSITIONS FOR SUPPLEMENTING PRODUCTS WITH THERAPEUTIC AGENTS AND METHOD OF USE THEREOF

(71) Applicant: DISRUPTION LABS, INC., Austin, TX (US)

(72) Inventors: Adam Garland, Austin, TX (US); Brian R. SLOAT, Austin, TX (US)

(21) Appl. No.: 18/767,811

(22) Filed: Jul. 9, 2024

Related U.S. Application Data
(60) Provisional application No. 63/525,777, filed on Jul. 10, 2023.

(51) Int. Cl. A61K 47/14; A61K 45/06; A61K 47/24; A61K 47/36

(52) U.S. Cl. CPC A61K 47/14; A61K 45/06; A61K 47/24; A61K 47/36

(57) ABSTRACT

Several embodiments pertain to nanoparticle-based compositions and their use in methods for the delivery of active agents to subjects and to products. In several embodiments, the compositions are stable for prolonged periods of time and under hot conditions and provide enhanced bioavailability and/or dispersibility.

### I. CROSS REFERENCE TO RELATED APPLICATIONS

The present application claims the benefit of priority of U.S. Provisional Patent Application No. 63/525,777, filed Jul. 10, 2023. The contents of the referenced application are incorporated into the present application by reference.

### BACKGROUND

### II. Field of the Disclosure

This disclosure relates to the fields of nanoparticles, drug delivery, and medicine.

### III. Background

Currently, a number of hydrophobic compounds are used as pharmaceuticals, nutraceuticals, cosmetics, dietary supplements, food additives, fragrances, etc. Pure isolate forms of plant extracts may be highly hydrophobic and have different characteristics than their impure counterparts from a formulation and pharmacokinetic standpoint. Use of hydrophobic compounds to treat, prevent, or cure disease and conditions has drawbacks such as poorer bioavailability due to low solubility in water.

Attempts have been made to produce delivery systems for hydrophobic compounds. However, delivery systems for active ingredients can also suffer from a variety of problems. For example, current nanoparticle technology can often be too unstable for effective use.

Current nanoparticle formulations may distribute unevenly through a solution or a liquid medium, which may cause uneven delivery of the nanoparticle. The uneven distribution may cause the nanoparticles to form a layer or precipitate out of a solution. Current nanoparticles may also separate over time. The distribution of nanoparticles and/or the active ingredients within current nanoparticle formulation may also suffer from instability.

Several embodiments disclosed herein solve these or other problems by providing novel and innovative nanoparticle compositions and methods for generating such compositions.

### BRIEF SUMMARY

Certain aspects of the disclosure comprise a nanoparticle composition, comprising a nanoparticle comprising a first active agent at a weight percent in the composition ranging from 0.001% to 20%; a lipid source at a weight percent in the composition ranging from 1% to 50%; optionally a surfactant at a weight percent in the composition ranging from 0% to 17.5%; and water at a weight percent in the composition ranging from 50% to about 97.5%. In some instances, the nanoparticles have an average size ranging from about 25 nm to about 200 nm. In some aspects, the composition is a dried composition comprising a nanoparticle having weight ratios of a first active agent: a lipid source: and optionally a surfactant of 0.002 to 50:2 to 87.5:0 to 17.5. In some aspects, the active agent is a pharmaceutical, nutraceutical, cosmetic, pigment, flavoring, etc. In some aspects, the first active agent is hydrophobic. In some aspects, the first active agent is coenzyme Q10, a vitamin E, a beta carotene, squalene, a vitamin K, a docosahexaenoic acid, a curcuminoid, a phytoceramide, vitamin D3, and/or an ashwagandha extract. It has been found that the nanoparticle compositions can provide stability over months and/or shelf life of over months for the active agents and/or components therein and/or the size of the particle. In some aspects, the nanoparticles further comprise one or more gums, such as xanthan gum and/or a biosaccharide gum. It has been found that the nanoparticle compositions comprising one or more gums have an increased stability in aqueous liquids at temperatures over 70° C. as compared to nanoparticles without a gum.

Certain aspects of the disclosure comprise a nanoparticle composition, comprising a nanoparticle comprising a lipid source at a weight percent in the composition ranging from 1% to 50%; optionally a surfactant at a weight percent in the composition ranging from 0% to 17.5%; optionally a gum at a weight percent in the composition ranging from 0% to 5%; and water at a weight percent in the composition ranging from 50% to about 97.5%. In some instances, the nanoparticles have an average size ranging from about 25 nm to about 200 nm. In some aspects, the composition is a dried composition comprising a nanoparticle having weight ratios of a lipid source and optionally a surfactant of 2 to 87.5:0 to 17.5. It has been found that the nanoparticle compositions can provide stability over months and/or shelf life of over months for the components therein and/or the size of the particle. It has been found that the nanoparticle compositions that contain a gum can provide increased stability in hot aqueous liquids with temperatures over 70° C.

In some aspects, the composition is configured such that when concentrated to dryness to afford a powder formulation of nanoparticles, the nanoparticle powder can be reconstituted to provide the nanoparticle composition. Disclosed herein, it has been found that modifying the mixtures of nanoparticles (e.g., liposome, micelle, nanoemulsion, multi-lamellar, double liposome, solid lipid particles) and/or density of the mixture of particles can provide increased stability to the actives, stability to the particles, and can be tuned for the particular liquid or carrier that the mixture is being added to for better dispersion and more stable dispersion.

Certain aspects of the disclosure comprise a fortified biomass comprising a biomass coated with the nanoparticle composition of any aspect disclosed herein. In some aspects, the biomass is a plant biomass, such as a hemp biomass, marijuana biomass, a moonrock, hash, mushroom biomass, kratom biomass, kana biomass, and/or kava biomass.

Certain aspects of the disclosure comprise a method of treating a patient in need of treatment comprising administering an effective amount of the nanoparticle composition of any aspect disclosed herein or the fortified biomass of any aspect disclosed herein to the patient.

Certain aspects of the disclosure comprise a method of manufacturing a nanoparticle composition of an active agent comprising providing a lipid source; optionally providing a surfactant; mixing the lipid source and optionally the surfactant to provide a solution; passing the solution through a microfluidizer to provide a mixed nanoparticle composition; and mixing an active agent with the mixed nanoparticle composition. In some aspects, the method further comprises adding one or more co-emulsifiers to the solution. In some aspects, the method further comprises adding water to the solution.

In some aspects, the nanoparticle compositions of the present invention can include nanoparticles dispersed in a liquid phase (e.g., aqueous solution). The liquid phase can include dispersed components (e.g., nanoparticles or other non-solubilized components) and/or solubilized components (e.g., salts, acids, etc.).
Aspect 1 is directed to a nanoparticle composition comprising water at about 60 wt. % to about wt. 93.799% and at least one nanoparticle, the nanoparticle comprising:
   at least one active agent at 0.001 wt. % to 20 wt. %;
   at least one phospholipid at 5 wt. % to 20 wt. %;
   at least one triglyceride at 1 wt. % to 15 wt. %;
   at least one sterol at 0.2 wt. % to 2 wt. %; and
   optionally at least one surfactant at 0 wt. % to 17.5 wt. %,
      wherein the weight percentages are based on the weight of the composition.
Aspect 2 is directed to the nanoparticle composition of Aspect 1, wherein the active agent comprises one or more pharmaceutical, nutraceutical, cosmetic, pigment, or flavoring.
Aspect 3 is directed to the nanoparticle composition of any one of Aspects 1 to 2, wherein the active agent comprises a hydrophobic active ingredient.
Aspect 4 is directed to the nanoparticle composition of any one of Aspects 1 to 3, wherein the active agent comprises coenzyme Q10, a vitamin E, a beta carotene, squalene, a vitamin K, a docosahexaenoic acid, a curcuminoid, a phytoceramide, vitamin D3, and/or an ashwagandha extract.
Aspect 5 is directed to the nanoparticle composition of Aspect 4, wherein the vitamin K comprises a powdered vitamin K or a vitamin K oil.
Aspect 6 is directed to the nanoparticle composition of any one of Aspects 1 to 5, wherein the active agent comprises a small molecule.
Aspect 7 is directed to the nanoparticle composition of any one of Aspects 1 to 6, wherein the active agent comprises a biologic.
Aspect 8 is directed to the nanoparticle composition of any one of Aspects 1 to 7, wherein the active agent comprises a flavoring agent.
Aspect 9 is directed to the nanoparticle composition of any one of Aspects 1 to 8, wherein the active agent comprises a cosmetic.
Aspect 10 is directed to the nanoparticle composition of any one of Aspects 1 to 9, wherein the triglyceride comprises a medium chain triglyceride.
Aspect 11 is directed to the nanoparticle composition of any one of Aspects 1 to 10, wherein the phospholipid comprises a lysophospholipid, a phosphatidylcholine, and/or a phosphatidylserine.
Aspect 12 is directed to the nanoparticle composition of any one of Aspects 1 to 11, wherein a source of the phospholipid is 20% pure, 50%, pure, or 90% pure.
Aspect 13 is directed to the nanoparticle composition of any one of Aspects 1 to 12, wherein the phospholipid comprises hydrogenated soybean phosphatidylcholine and/or sunflower phosphatidylcholine.
Aspect 14 is directed to the nanoparticle composition of any one of Aspects 1 to 13, wherein the surfactant comprises an emulsifier.
Aspect 15 is directed to the nanoparticle composition of any one of Aspects 1 to 14, wherein the at least one nanoparticle does not comprise a surfactant.
Aspect 16 is directed to the nanoparticle composition of any one of Aspects 1 to 15, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.
Aspect 17 is directed to the nanoparticle composition of any one of Aspects 1 to 16, comprised in a liquid formulation.
Aspect 18 is directed to the nanoparticle composition of Aspect 17, wherein the density of the nanoparticle composition is within 10% of the density of the liquid formulation.
Aspect 19 is directed to the nanoparticle composition of any one of Aspects 1 to 18, further comprising at least one co-emulsifier and/or at least one preservative.
Aspect 20 is directed to the nanoparticle composition of Aspect 19, wherein the co-emulsifier is a gum.
Aspect 21 is directed to the nanoparticle composition of Aspect 20, wherein the gum is a xanthan gum and/or a biosaccharide gum.
Aspect 22 is directed to the nanoparticle composition of any one of Aspects 20 to 21, wherein the composition comprises 0.01 wt. % to 5 wt. % of the gum, wherein the weight percentages are based on the weight of the composition.
Aspect 23 is directed to the nanoparticle composition of any one of Aspects 1 to 22, wherein the at least one nanoparticle or the nanoparticle composition comprises:
   at least one triglyceride comprising capric and caprylic triglycerides;
   DL alpha tocopherol;
   a plant sterol and/or cholesterol;
   optionally xanthan gum; and
   optionally hydrogenated phosphatidylcholine.
Aspect 24 is directed to a nanoparticle composition comprising water at about 60 wt. % to about wt. 93.799% and at least one nanoparticle, the nanoparticle comprising:
   at least one phospholipid at 5 wt. % to 20 wt. %;
   at least one triglyceride at 1 wt. % to 15 wt. %;
   at least one sterol at 0.2 wt. % to 2 wt. %; and
   optionally at least one surfactant at 0 wt. % to 17.5 wt. %,
      wherein the weight percentages are based on the weight of the composition.
Aspect 25 is directed to the nanoparticle composition of Aspect 24, wherein the triglyceride comprises a medium chain triglyceride.
Aspect 26 is directed to the nanoparticle composition of any one of Aspects 24 to 25, wherein the phospholipid comprises a lysophospholipid, a phosphatidylcholine, and/or a phosphatidylserine.
Aspect 27 is directed to the nanoparticle composition of any one of Aspects 24 to 26, wherein a source of the phospholipid is 20% pure, 50%, pure, or 90% pure.
Aspect 28 is directed to the nanoparticle composition of any one of Aspects 24 to 27, wherein the phospholipid comprises hydrogenated soybean phosphatidylcholine and/or sunflower phosphatidylcholine.
Aspect 29 is directed to the nanoparticle composition of any one of Aspects 24 to 28, wherein the surfactant comprises an emulsifier.
Aspect 30 is directed to the nanoparticle composition of any one of Aspects 24 to 29, wherein the at least one nanoparticle does not comprise a surfactant.
Aspect 31 is directed to the nanoparticle composition of any one of Aspects 24 to 30, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.
Aspect 32 is directed to the nanoparticle composition of any one of Aspects 24 to 31, comprised in a liquid formulation.
Aspect 33 is directed to the nanoparticle composition of Aspect 32, wherein the density of the nanoparticle composition is within 10% of the density of the liquid formulation.
Aspect 34 is directed to the nanoparticle composition of any one of Aspects 24 to 33, further comprising at least one co-emulsifier and/or at least one preservative.
Aspect 35 is directed to the nanoparticle composition of Aspect 34, wherein the co-emulsifier is a gum.
Aspect 36 is directed to the nanoparticle composition of Aspect 35, wherein the gum is a xanthan gum and/or a biosaccharide gum.
Aspect 37 is directed to the nanoparticle composition of any one of Aspects 35 to 36, wherein the composition comprises 0.01 wt. % to 5 wt. % of the gum, wherein the weight percentages are based on the weight of the composition.
Aspect 38 is directed to the nanoparticle composition of any one of Aspects 24 to 37, wherein the at least one nanoparticle or nanoparticle composition comprises:
   at least one triglyceride comprising capric and caprylic triglycerides;
   DL alpha tocopherol;
   a plant sterol and/or cholesterol;
   optionally xanthan gum; and
   optionally hydrogenated phosphatidylcholine.
Aspect 39 is directed to a method of manufacturing a nanoparticle composition of any one of Aspects 1 to 38, the method comprising the steps of:
   (a) adding one or more phospholipid, one or more triglyceride, one or more sterol, optionally one or more active agents, and optionally one or more surfactants to water;
   (b) mixing the ingredients of step (a) creating a mixture;
   (c) homogenizing the mixture creating a homogenized mixture;
   (d) performing microfluidization on the homogenized mixture creating a microfluid;
   (e) sonicating the microfluid creating a sonicated microfluid;
   (f) stirring the sonicated microfluid creating a stirred microfluid;
   (g) creating a coacervation from the stirred microfluid; and
   (h) precipitating the coacervation.
Aspect 40 is directed to the method of Aspect 39, wherein the mixing of step (b) comprises high sheer mixing.
Aspect 41 is directed to the method of any one of Aspects 39 to 40, wherein the homogenizing of step (c) comprises high pressure homogenization.
Aspect 42 is directed to the method of any one of Aspects 39 to 41, wherein the stirring of step (f) comprises mechanical stirring.
Aspect 43 is directed to the method of any one of Aspects 39 to 42, wherein the precipitating of step (h) comprises solvent precipitation.
Aspect 44 is directed to the method of any one of Aspects 39 to 43, further comprising extruding the composition using hot melt extrusion.
Aspect 45 is directed to the method of any one of Aspects 39 to 44, further comprising drying the nanoparticle composition.
Aspect 46 is directed to the method of Aspect 45, wherein the drying comprises lyophilizing, spray drying, fluid bed drying, and/or desiccating the nanoparticle composition.
Aspect 47 is directed to a nanoparticle composition comprising water at about 0 wt. % to about wt. 10% and at least one nanoparticle, the nanoparticle or nanoparticle composition comprising:
   optionally at least one active agent at 0.002 wt. % to 50 wt. %;
   at least one phospholipid at 10 wt. % to 50 wt. %;
   at least one triglyceride at 2 wt. % to 37.5 wt. %;
   at least one sterol at 0.4 wt. % to 5 wt. %;
   optionally at least one gum at 0.01 wt. % to 5 wt. %; and
   optionally at least one surfactant at 0 wt. % to 43.75 wt. %,
Aspect 48 is directed to the nanoparticle composition of Aspect 47, wherein the active agent comprises one or more pharmaceutical, nutraceutical, cosmetic, pigment, or flavoring.
Aspect 49 is directed to the nanoparticle composition of any one of Aspects 47 to 48, wherein the composition comprises a mixture of nanoparticles selected from at least two of a liposome, a micelle, a nanoemulsion, a multi-lamellar particle, a double liposome particle, and a solid lipid particle.
Aspect 50 is directed to the nanoparticle composition of any one of Aspects 47 to 49, wherein the at least one nanoparticle or nanoparticle composition comprises:
   at least one triglyceride comprising capric and caprylic triglycerides;
   DL alpha tocopherol;
   a plant sterol and/or cholesterol;
   optionally xanthan gum; and
   optionally hydrogenated phosphatidylcholine.
Aspect 51 is directed to a method of treating a disease, a disorder, and/or a symptom in an individual, the method comprising administering to the individual a therapeutically effective amount of the nanoparticle composition of any one of Aspects 1 to 38, 47 to 50, or 62.
Aspect 52 is directed to the method of Aspect 51, wherein the disease is an autoimmune disease, a cancer, a degenerative disease, a blood disease, an infection, and/or a deficiency disease.
Aspect 53 is directed to the method of any one of Aspects 51 to 52, wherein the symptom comprises opioid withdrawal, pain, anxiety, depression, insomnia, inflammation, fever, fatigue, muscle aches, or a combination thereof.
Aspect 54 is directed to the method of any one of Aspects 51 to 53, wherein the administering step comprises local administration.
Aspect 55 is directed to the method of any one of Aspects 51 to 54, wherein the administering step comprises systemic administration.
Aspect 56 is directed to the method of any one of Aspects 51 to 55, wherein the administering step comprises oral administration.
Aspect 57 is directed to the method of any one of Aspects 51 to 56, wherein the therapeutically effective amount of the nanoparticle composition comprises 10 mg/kg of the individual to 200 mg/kg of the individual.
Aspect 58 is directed to a method of distributing an active agent in a solution, the method comprising contacting the solution with the nanoparticle composition of any one of Aspects 1 to 38, 47 to 50, or 62.
Aspect 59 is directed to the method of Aspect 58, wherein the nanoparticle composition has a density within 10% of a density of the solution.
Aspect 60 is directed to the method of any one of Aspects 58 to 59, wherein the nanoparticle composition comprises a mixture of nanoparticles selected from at least two of a liposome, a micelle, a nano emulsion, a multi-lamellar particle, a double liposome particle, and a solid lipid particle.
Aspect 61 is directed to a method of adjusting a density of a nanoparticle composition, the method comprising adjusting the density of the nanoparticle composition of any one of Aspects 1 to 38 or 47 to 50 by adjusting a ratio of at least two of a liposome, a micelle, a nanoemulsion, a multi-lamellar particle, a double liposome particle, and a solid lipid particle.
Aspect 62 is directed to the nanoparticle composition of any one of Aspects 1-38 and 47-50, wherein the nanoparticle composition further optionally comprises potassium sorbate, sodium benzoate, and one or both of citric acid and/or ascorbic acid. The nanoparticle composition can include the nanoparticles dispersed in a liquid phase (e.g., aqueous solution). The liquid phase can include the potassium sorbate, sodium benzoate, and optionally one or both of citric acid and/or ascorbic acid. The potassium sorbate, sodium benzoate, and the optional citric acid and/or ascorbic acid can be dissolved in the liquid phase.

It is contemplated that any embodiment discussed in this specification can be implemented with respect to any method or composition of the disclosure, and vice versa. Furthermore, compositions of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is a flow chart showing an embodiment of a method of preparing a composition as disclosed herein.

FIG. 2 is a flow chart showing another embodiment of a method for preparing a composition as disclosed herein.

### DETAILED DESCRIPTION

Disclosed herein are nanoparticle compositions, comprising a nanoparticle comprising a first active agent at a weight percent in the composition ranging from 0.001% to 20%; a lipid source at a weight percent in the composition ranging from 1% to 50%; optionally a surfactant at a weight percent in the composition ranging from 0% to 17.5%; optionally a gum at a weight percent in the composition ranging from 0% to 5%; and water at a weight percent in the composition ranging from 50% to about 97.5%. In some instances, the nanoparticles have an average size ranging from about 25 nm to about 200 nm. In some aspects, the composition is a dried composition comprising a nanoparticle having weight ratios of a first active agent: a lipid source: and a optionally a surfactant of 0.002 to 50:2 to 87.5:0 to 17.5. It has been found that compositions disclosed herein can be configured such that when concentrated to dryness to afford a powder formulation of nanoparticles, the nanoparticle powder can be reconstituted to provide the nanoparticle composition. It has been found that the nanoparticle compositions can provide stability over months and/or shelf life of over months for the active agents and/or components therein and/or the size of the particle. It has been found that the nanoparticle compositions that contain a gum can provide increased stability in hot aqueous liquids with temperatures over 70° C.

Disclosed herein are nanoparticle compositions comprising a nanoparticle comprising a lipid source at a weight percent in the composition ranging from 1% to 50%; optionally a surfactant at a weight percent in the composition ranging from 0% to 17.5%; optionally a gum at a weight percent in the composition ranging from 0% to 5%; and water at a weight percent in the composition ranging from 50% to about 97.5%. In some instances, the nanoparticles have an average size ranging from about 25 nm to about 200 nm. In some aspects, the composition is a dried composition comprising a nanoparticle having weight ratios of a lipid source and optionally a surfactant of 2 to 87.5:0 to 17.5. It has been found that the nanoparticle compositions can provide stability over months and/or shelf life of over months for the components therein and/or the size of the particle. It has been found that the nanoparticle compositions that contain a gum can provide increased stability in hot aqueous liquids with temperatures over 70° C.

Also, it has been found that modifying the mixtures of nanoparticles (e.g., liposome, micelle, nanoemulsion, multi-lamellar, double liposome, solid lipid particles) and/or density of the mixture of particles can provide increased stability to the actives, stability to the particles, and can be tuned for the particular liquid or carrier that the mixture is being added to for better dispersion and more stable dispersion. Applicant has found that compositions as described herein can contain mixtures of nanoparticles (e.g., liposome, micelle, nanoemulsion, multi-lamellar, double liposome, solid lipid particles) and that the mixture and/or density of the mixture can be modified.

### I. Definitions

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. When used in the context of a compound, composition or device, the term “comprising” means that the compound, composition or device includes at least the recited features or components but may also include additional features or components.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed disclosure.

Unless otherwise defined, all technical and scientific terms used herein have the same meaning as commonly understood by one of ordinary skill in the art to which this subject matter belongs. The terminology used in the description of the subject matter herein is for the purpose of describing particular embodiments only and is not intended to be limiting of the subject matter.

The terms “treatment,” “treating,” “treat” and the like shall be given its ordinary meaning and shall also include herein to generally refer to obtaining a desired pharmacologic and/or physiologic effect. The effect may be prophylactic in terms of completely or partially preventing a disorder, disease, or symptom thereof and/or may be therapeutic in terms of a partial or complete stabilization or cure for a disorder or disease and/or adverse effect attributable to the disorder or disease. “Treatment” shall also cover any treatment of a disorder or disease in a mammal, particularly a human, and includes: (a) preventing the disorder, disease, or symptom (e.g., of the disorder or disease) from occurring in a subject which may be predisposed to the disorder, disease, or symptom but has not yet been diagnosed as having it; (b) inhibiting the disorder, disease, or symptom, e.g., arresting its development; and/or (c) relieving the disorder, disease, or symptom (e.g., causing regression of the disorder, disease, or symptom).

The “patient” or “subject” treated as disclosed herein may be a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, mini-pigs (a mini-pig is a small breed of swine weighing about 35 kg as an adult), horses, cats, dog, rabbits, rodents (e.g., rats or mice), monkeys, etc. Human subjects include neonates, infants, children, juveniles, adults and geriatric subjects. The subject can be a subject “in need of” the methods disclosed herein can be a subject that is experiencing a disease state and/or is anticipated to experience a disease state, and the methods and compositions of the disclosure are used for therapeutic and/or prophylactic treatment.

As used herein, the terms “active agent”, “active compound”, “pharmaceutical composition”, “therapeutic agent”, and the like may be used interchangeably. The terms generally refer to compositions having pharmacological activity or other direct effects in the diagnosis, cure, mitigation, treatment, or prevention of disease, or to affect the structure, appearance, or any function of molecules, cells, tissues, organs, or subject. The terms may refer to compositions in a beverage. The terms may refer to pharmaceuticals, nutraceuticals, cosmetics, pigments, flavorings, and the like. The terms may refer to compositions that are hydrophobic, hydrophilic, or both.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, ascorbic acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in U.S. Pat. No. 4,783,443A, Johnston et al., published Sep. 11, 1987 (incorporated by reference herein in its entirety).

An “effective amount” or a “therapeutically effective amount” as used herein can refer to an amount of a therapeutic agent that is effective to relieve, to some extent, or to reduce the likelihood of onset of, one or more of the symptoms of a disease or condition (e.g., disorder), and includes curing a disease or condition. “Curing” means that the symptoms of a disease or condition are eliminated; however, certain long-term or permanent effects may exist even after a cure is obtained (such as extensive tissue damage).

As used herein, the term “weight percent” (or wt. %, weight %, percent by weight, etc.), when referring to a component, is the weight of the component divided by the weight of the composition that includes the component, multiplied by 100%. For example, the weight percent of component A when 5 grams of component A is added to 95 grams of component B is 5% (e.g., 5 g A/(5 g A+95 g B)×100%).

As used herein, the “dry weight %” (e.g., “dry wt. %”, “dry weight percent”, etc.) of an ingredient is the weight percent of that ingredient in the composition where the weight of water has not been included in the calculation of the weight percent of that ingredient. A dry weight % can be calculated for and includes either a composition that does not include water (e.g., that has been dried to, for example, a powder) or for a composition that includes water but where the amount of water is not included in the calculation.

As used herein, the “wet weight %” (e.g., “wet wt. %”, “wet weight percent”, etc.) of an ingredient is the weight percent of that ingredient in a composition where the weight of water is included in the calculation of the weight percent of that ingredient. For example, the dry weight percent of component A when 5 grams of component A is added to 95 grams of component B and 100 grams of water is 5% (e.g., 5 g A/(5 g A+95 g B)×100%). Alternatively, the wet weight percent of component A when 5 grams of component A is added to 95 grams of component B and 100 grams of water is 2.5% (e.g., 5 g A/(5 g A+95 g B+100 g water)×100%).

As used herein, the term “weight volume percent” (or weight volume percentage, w/v, w/v (%), etc.), when referring to a component, is the weight of the component in grams divided by the volume of a solution in milliliters that includes the component, multiplied by 100%. For example, the w/v of component A when 5 grams of component A is added to a solution to provide 100 mL of solution is 5 w/v (%) (e.g., 5 g solute A/100 mL solution×100%).

When referring to an amount present for one or more ingredients, the term “collectively or individually” (and variations thereof) means that the amount is intended to signify that the ingredients combined may be provided in the amount disclosed, or each individual ingredient may be provided in the amount disclosed. For example, if agents A and B are referred to as collectively or individually being present in a composition at a wt. % of 5%, that means that A may be at 5 wt. % in the composition, B may be at 5 wt. % in the composition, or the combination of A and B may be present at a total of 5 wt. % (A+B=5 wt. %). Alternatively, where both A and B are present, A may be at 5 wt. % and B may be at 5 wt. %, totaling 10 wt. %.

When referring to the amount present for one or more ingredients, the terms “or ranges including and/or spanning the aforementioned values” (and variations thereof) is meant to include any range that includes or spans the aforementioned values. For example, when the wt. % of an ingredient is expressed as 1%, 5%, 10%, 20%, “or ranges including and/or spanning the aforementioned values,” this includes wt. % ranges for the ingredient spanning from 1% to 20%, 1% to 10%, 1% to 5%, 5% to 20%, 5% to 10%, and 10% to 20%.

As used herein, “polydispersity” or “PDI” is used to describe the degree of non-uniformity of a size distribution of particles. Also known as the heterogeneity index, PDI is a number calculated from a two-parameter fit to the correlation data (the cumulants analysis). This index is dimensionless and scaled such that values smaller than 0.05 are mainly seen with highly monodisperse standards.

As used herein, the term “phytocannabinoids” refers to a group of cannabinoids that occur naturally in the cannabis plant, including but not limited to, THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran).

As used herein, the term “cannabinoid” refers to the chemical substance, regardless of structure or origin, that joins the cannabinoid receptors of the body and brain and that have similar effects to those produced by the cannabis plant. As used herein, the term “cannabinoid” includes but is not limited to Cannabichromenes (e.g., cannabichromene (CBC), cannabichromenic acid (CBCA), cannabichromevarin (CBCV), cannabichromevarinic acid (CBCVA), Cannabicyclols (e.g., cannabicyclol (CBL), cannabicyclolic acid (CBLA), cannabicyclovarin (CBLV), etc.), Cannabidiols (e.g., cannabidiol (CBD), cannabidiol monomethylether (CBDM), cannabidiolic acid (CBDA), Cannabidiol-C₄ (CBD-C₄), cannabidiorcol (CBD-C1), cannabidivarin (CBDV), cannabidivarinic acid (CBDVA), etc.), Cannabielsoins (e.g., Cannabielsoic Acid (CBEA), Cannabielsoic acid A (CBEA-A), cannabielsoic acid B (CBEA-B), cannabielsoin (CBE), cannabielsoin acid A (CBEA-A), etc.), Cannabigerols (e.g., cannabigerol (CBG), cannabigerol monomethylether (CBGM), cannabigerolic acid (CBGA), cannabigerolic acid monomethylether (CBGAM), cannabigerovarin (CBGV), cannabigerovarinic acid (CBGVA), etc.), Cannabinols and cannabinodiols (e.g., cannabinodiol (CBND), cannabinodivarin (CBVD), cannabinol (CBN) cannabinol methylether (CBNM), cannabinol-C2 (CBN-C2), cannabinol-C4 (CBN-C4), cannabinolic acid (CBNA), cannabiorcool (CBN-C1), cannabivarin (CBV), etc.), Cannabitriols (e.g., 10-ethoxy-9-hydroxy-delta-6a-tetrahydrocannabinol, 8,9-dihydroxy-delta-61-tetrahydrocannabinol, 8,9-Dihydroxy-Δ^{6a(10a)}-tetrahydrocannabinol (8,9-Di-OH-CBT-C₅), cannabitriol (CBT), cannabitriolvarin (CBTV), Ethoxy-cannabitriolvarin (CBTVE), etc.), tetrahydrocannabinols (e.g., tetrahydrocannabinol (THC), tetrahydrocannabinol-C4 (THC-C4), delta-9-tetrahydrocannabinol (Δ9-THC), delta-9-tetrahydrocannabinol-C4 (Δ9-THC-C4), delta-9-tetrahydrocannabinolic acid A (THCA-A), Tetrahydrocannabinolic Acid (THCA), delta-9-tetrahydrocannabinolic acid B (THCA-B) delta-9-tetrahydrocannabinolic acid-C4 (THCA-C4), Tetrahydrocannbinol C₄ (THC-C₄), delta-9-tetrahydrocannabiorcol (THC-C1), delta-9-tetrahydrocannabiorcolic acid (THCA-C1), delta-9-tetrahydrocannabivarin (THCV), delta-9-tetrahydrocannabivarinic acid (THCVA), delta-9-cis-tetrahydrocannabinol (cis-THC), trihydroxy-delta-9-tetrahydrocannabinol (triOH-THC), etc.), and/or other cannabinoids (e.g., 10-Oxo-delta-6a-tetrahydrocannabinol (OTHC), cannabichromanon (CBCF), cannbifuran (CBF), cannabiglendol, cannabiripsol (CBR), cannbicitran, dehydrocannabifuran (DCBF), 3,4,5,6-tetrahydro-7-hydroxy-alpha-alpha-2-trimethyl-9-n-propyl-2,6-methano-2H-1-benzoxocin-5-methanol (OH-iso-HHCV), Δ⁷-cis-iso-tetrahydrocannabivarin, Δ⁸-tetrahydrocannabinolic acid (Δ8-THCA), tetrahydrocannabiorcolic acid (THCA-C₄), Cannabivarinodiolic (CBNDVA), Cannabivarinodiol (CBNDV), Δ⁸-tetrahydrocannabinol (Δ⁸-THC), Cannabivarinselsoin (CBEV), Cannabivarinselsoinic Acid (CBEVA), Cannabielvarinsoin (CBLV), Cannabiclvarinsoinic Acid (CBLVA), Cannabivarinic Acid (CBNVA), Cannabiorcol (CBN-C₁), Cannabinodiolic Acid (CBNDA), and/or 3,4,5,6-tetrahydro-7-hydroxy-alpha-alpha-2-trimethyl-9-n-propyl-2,6-methano-2H-1-benzoxocin-5-methanol (OH-iso-HHCV). Cannabinoids can also include cannabinoids derived from sources other than hemp or marijuana, such as oranges. Cannabinoids can also include synthetic (e.g., not naturally occurring) chemicals.

As used herein, the term “hydrophobic”, “water insoluble”, or “insoluble in water”, or “not soluble in water”, or similar terms can refer to a chemical that has a water solubility below 0.1 g in 100 ml of water at 20° C. and 101.325 kPa of pressure. As used herein, the term “hydrophilic”, or “water soluble”, or “soluble in water”, or similar terms can refer to a chemical that has a water solubility at or above 1 g in 100 ml of water at 20° C. and 101.325 kPa of pressure. As used herein, the term “semi-soluble in water”, “water semi-soluble”, or “semi-insoluble in water”, or “water semi-insoluble”, or similar terms can refer to a chemical that has a water solubility at 0.1 g in 100 ml of water at 20° C. and 101.325 kPa of pressure to below 1 g in 100 ml of water at 20° C. and 101.325 kPa of pressure.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of any embodiment disclosed herein, but instead as merely intended to highlight alternative or additional features that may or may not be utilized in a particular embodiment of the disclosure. Likewise, a group of items linked with the conjunction “or” should not be read as requiring mutual exclusivity among that group, in some embodiments, but rather should be read as “and/or”.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein. In this application, the use of the singular includes the plural unless specifically stated otherwise.

### II. Compositions

Certain embodiments disclosed herein concern compositions comprising a nanoparticle, which may encapsulate an active agent. The compositions, in some embodiments, can deliver highly pure active agents, such as pharmaceuticals, nutraceuticals, cosmetics, pigments, flavorings, etc. in a nanoparticle delivery system (e.g., lipid nanoparticle, a liposomal system, oil-in-water emulsions, dry liposome particles, etc.). Some active agents include hydrophobic active agents. Some nanoparticles do not comprise an active agent. Some active agents include, but are not limited to coenzyme Q10, a vitamin E, a beta carotene, squalene, a vitamin K, a docosahexaenoic acid, a curcuminoid, a phytoceramide, vitamin D3, and/or an ashwagandha extract. Compositions disclosed herein, in some embodiments, comprise mixed nanoparticle compositions comprising active agents or combinations of active agents. In several embodiments, the disclosed compositions, which may be mixed nanoparticle compositions, have certain characteristics including, but not limited to, fewer impurities, fewer variations batch-to-batch (e.g., stability, degradation profiles, efficacy), better delivery predictability, fewer side effects when administered to a subject, higher bioavailability, faster onset of activity, greater long term storage stability of the particles and the active ingredient(s), greater stability at higher temperatures of the particles and the active ingredient(s), better dispersibility, greater stability of a dispersion, and better efficacy, relative to the characteristics of compositions known in the art.

Certain embodiments concern nanoparticles, including mixed micelle-based compositions, and their use in methods for the delivery of active compounds, which may include plant extracts and/or other beneficial agents (e.g., vitamins, nutrients, other plant extracts, nutraceuticals, pharmaceuticals, flavorings, pigments, or other beneficial agents for delivery). In several embodiments, the compositions are stable (e.g., at room temperature) for prolonged periods of time.

Several embodiments disclosed herein pertain to formulations, including mixed nanoparticle compositions, for the delivery of one or more active agents (e.g., active agents) to subjects. Several embodiments pertain to methods of use and making the composition. In several embodiments, the nanoparticle compositions comprise one or more active agents (e.g., single active agents or combinations thereof). In several embodiments, the composition is comprised of high-quality, pure, and/or high-grade ingredients (e.g., highly pure) that yield a well-characterized, reproducible delivery system (e.g., comprising mixed nanoparticles). In several embodiments, the compositions have enhanced stability (e.g., are stable for long periods of time under various conditions). In several embodiments, the composition confers water solubility to hydrophobic agents, to combinations of hydrophobic agents, and/or to combinations of hydrophobic and hydrophilic agents. In several embodiments, the nanoparticle composition comprises a liposomal and/or nano-emulsion composition of an active agent.

While some embodiments are disclosed herein in relation to particular active agents, it is to be understood that other active agents, nutrients, and/or combinations thereof can be employed in the compositions disclosed herein. In several embodiments, for example, hydrophilic active agents may also be provided in the disclosed nanoparticle compositions (e.g., alone, in combination with other hydrophilic active agents, and/or in combination with hydrophobic active agents). Advantageously, the compositions disclosed herein may enhance the delivery of and/or slow or lessen the degradation of hydrophilic or hydrophobic agents (or combinations thereof). Additionally, while some embodiments are disclosed in relation to nanoparticles (e.g., mixed micelle-based nanoparticles), as disclosed elsewhere herein, microparticles are also envisioned.

Certain embodiments disclosed herein comprise nanoparticle products, which may comprise active compositions. In several embodiments, the composition comprises a nanoparticle delivery system, which may be utilized to impart apparent aqueous solubility and deliverability to an otherwise practically water insoluble molecule. Attributes of some embodiments disclosed herein have been determined to be high quality and reproducible. Such reproducibility and low variations may allow the products to generate a reproducible certificate of analysis for different batches.

In several embodiments, the compositions disclosed herein (e.g., mixed nanoparticle compositions and/or formulations comprising them) increase the bioavailability of active agents (e.g., pharmaceutical, nutraceutical, etc.), decrease the time for absorption of those active agents, increase the stability of the active agents or the particles comprising the active agents, increase the consistency of delivery (e.g., by limiting batch-to-batch variation), and/or increase the efficacy of the active agents (higher dosing and/or faster onset of activity).

As disclosed elsewhere herein, in some embodiments, the compositions (including the mixed nanoparticle compositions) disclosed herein are able to deliver active agents that are highly pure. In several embodiments, an active agent (e.g., pharmaceuticals, nutraceuticals, cosmetics, pigments, flavorings, and the like) formulated with the nanoparticle has a purity of greater than or equal to about: 80%, 90%, 95%, 98%, 99%, 99.5%, 99.9%, 99.99%, or ranges including and/or spanning the aforementioned values.

As disclosed herein, some embodiments relate to delivery systems (e.g., mixed nanoparticle compositions and/or formulations comprising the same) that improve the absorption of the highly insoluble forms of an active agent (e.g., pharmaceuticals, nutraceuticals, cosmetics, pigments, flavorings, and the like) or combination of agents. In several embodiments, the active agent encapsulated in the nanoparticle compositions disclosed herein (e.g., the starting material) has an aqueous solubility of less than or equal to about: 0.05 mg/mL, 0.01 mg/mL, 0.012 mg/mL, 0.001 mg/mL, or ranges including and/or spanning the aforementioned values, such as a range between 0.05 mg/mL to 0.001 mg/mL. In several embodiments, where a combination of active agents is encapsulated in the nanoparticle compositions disclosed herein, one or more or all of the active agents in the composition may have an aqueous solubility of less than or equal to about: 0.05 mg/mL, 0.01 mg/mL, 0.012 mg/mL, 0.001 mg/mL, or ranges including and/or spanning the aforementioned values, such as a range between 0.05 mg/mL to 0.001 mg/mL. In several embodiments, the aqueous solubility of the active agent or agents (and/or the amount of active agent or agents that can be provided in an aqueous solution) can be improved to equal to or greater than about: 1 mg/mL, 5 mg/mL, 10 mg/mL, 20 mg/mL, 30 mg/mL, 50 mg/mL, 100 mg/mL, or ranges including and/or spanning the aforementioned values. For example, the aqueous solubility of the active agent or agents may be increased to 1 mg/mL to 50 mg/mL, 10 mg/mL to 100 mg/mL, 1 mg/mL to 20 mg/mL, etc.

In several embodiments, at least one active agent in the nanoparticle composition (and/or combination of active agents provided in the mixed nanoparticle composition) is hydrophobic. In several embodiments, at least one hydrophobic active agent used to prepare a nanoparticle composition as disclosed herein (e.g., pharmaceuticals, nutraceuticals, cosmetics, pigments, flavorings, and the like) has an aqueous solubility of less than or equal to about: 0.05 mg/mL, 0.01 mg/mL, 0.012 mg/mL, 0.001 mg/mL, or ranges including and/or spanning the aforementioned values. In several embodiments, the solubility of the at least one active agent (e.g. the amount of the active agent that can be provided in an aqueous solution) used to prepare the compositions disclosed herein can be improved to equal to or greater than about: 1 mg/mL, 5 mg/mL, 20 mg/mL, 30 mg/mL, 50 mg/mL, 100 mg/mL, or ranges including and/or spanning the aforementioned values, when formulated with a nanoparticle. In several embodiments, the solubility of the at least one active agent can be improved by at least about: 50%, 100%, 150%, 200%, 500%, 1000%, 10,000%, or ranges including and or spanning the aforementioned values. In several embodiments, the solubility is measured as an amount that can be suspended for longer than 30 days and or that can be dissolved in an aqueous solution at a concentration of at least 20 mg/mL.

In several embodiments, as disclosed elsewhere herein, a nanoparticle composition (e.g., a mixed micelle composition, a liposomal composition, solid lipid particles, oil-in-water emulsions, water-in-oil-in-water emulsions, water-in-oil emulsions, oil-in-water-in-oil emulsions, etc.) is provided to aid in the delivery of active agents. As disclosed elsewhere herein, in several embodiments, the nanoparticles comprise one or more active agents. In several embodiments, one or more of the active ingredients is a nutraceutical. In several embodiments, a composition comprising the nanoparticles disclosed herein comprises therapeutically effective amount of one or more active ingredients. In several embodiments, the one or more active compounds comprise coenzyme Q10, a vitamin E, a beta carotene, squalene, a vitamin K, a docosahexaenoic acid, a curcuminoid, a phytoceramide, vitamin D3, an ashwagandha extract, or a combination of any of the foregoing.

In several embodiments, the active ingredients provided in the nanoparticle composition may comprise an unenriched extract (e.g., a mixture of agents as extracted from a single plant source), an enriched extract that has been enriched through purification processes (to have larger amounts of certain active agents), or any individual active component of the extract (e.g., a pure or substantially pure compound). As an example, the nanoparticle composition may include an unenriched extract that is isolated by bulk extraction of multiple actives from a plant biomass at one time. Alternatively, the nanoparticle composition may include actives that have been further processed to enrich the extract for particular active agents (e.g., having a higher wt. % of the active agent than un-processed extract). In several embodiments, alternatively, an active from an extract may be purified and may be pure and/or substantially pure, as disclosed elsewhere herein. For brevity, when a composition or particle disclosed herein is described as including plant extracts, the term “extracts” is meant to include any of the foregoing (e.g., including a full plant extract or partial plant extract that has not been enriched, an extract that has been enriched for particular components (e.g., particular active agents), and/or an extract that has been purified to provide, for example, highly pure individual components).

In several embodiments, the nanoparticle (or a composition comprising the nanoparticle) is composed and/or comprises one or more extracts from the same plant species. In several embodiments, the one or more extracts may be from any one or more plant strains.

In several embodiments, the nanoparticle (or compositions comprising the nanoparticle) comprises or is composed of plant powders and/or plant active ingredients (e.g., including but not limited to alkaloids). In other embodiments, the extracts may be produced synthetically (e.g., in a laboratory). In several embodiments, the synthetic extract may share a structure with an extract that is naturally occurring. In several embodiments, the extracts are analogs of natural extracts of a plant (e.g., produced synthetically).

In several embodiments, the plant extracts are isolated from their plant sources. In several embodiments, the plant extracts are isolated from their plant sources using solvent extraction. In several embodiments, the plant extracts are isolated from their plant sources using acid base titration. In several embodiments, the plant extracts are isolated from their plant sources using CO² (supercritical or nonsupercritical). In several embodiments, the plant extracts are isolated from their plant sources using cyrogenic ethanol. In several embodiments, the plant extracts are isolated from their plant sources using other forms of extraction. In several embodiments, the extract is an alkaloid, as disclosed elsewhere herein. In several embodiments, as disclosed elsewhere herein, the nanoparticle (or a composition comprising the nanoparticle) comprises or is composed of cannabinoids. In several embodiments, the nanoparticle does not comprise a cannabinoid. In several embodiments, the nanoparticle (or a composition comprising the nanoparticle) comprises or is composed of cannabinoids derived from resin or rosin (solventless extraction of cannabinoids achieved by pressing biomass). In several embodiments, the nanoparticle (or a composition comprising the nanoparticle) comprises or is composed of cannabinoids from a crude extract of hemp or marijuana (an extraction that is not further purified). In several embodiments, the lipid particle solution is composed of cannabinoids from combinations of sources, such as hemp oil fortified with cannabinoid isolate. The cannabinoids (including phytocannabinoids) may be any of the cannabinoids disclosed elsewhere herein and/or a mixture of one or more of such cannabinoids.

In several embodiments, the nanoparticle (or a composition comprising the nanoparticle) comprises a cannabichromene, a cannabicyclol, a cannabidiol, a cannabielsoin, a cannabigerol, a cannabinol, a cannabinodiol, a cannabitriol, a delta-9-tetrahydrocannabinol, another cannabinoid, a synthetic cannabinoid, and/or combinations of any of the foregoing. In several embodiments, the mixed nanoparticle composition comprises two or more cannabichromenes, cannabicyclols, cannabidiols, cannabielsoins, cannabigerols, cannabinols, cannabinodiols, cannabitriols, delta-9-tetrahydrocannabinols, other cannabinoids, synthetic cannabinoids, and/or combinations of any of the foregoing. In several embodiments, the nanoparticle composition comprises CBC, CBCA, CBCV, CBCVA, CBL, CBLA, CBLV, CBD, CBDM, CBDA, CBD-C1, CBDV, CBDVA, CBEA-B, CBE, CBEA-A, CBG, CBGM, CBGA, CBGAM, CBGV, CBGVA, CBND, CBVD, CBN, CBNM, CBN-C2, CBN-C4, CBNA, CBN-C1, CBV, 10-ethoxy-9-hydroxy-delta-6a-tetrahydrocannabinol, 8,9-dihydroxy-delta-61-tetrahydrocannabinol, CBT, CBTV, THC, THC-C4, THCA-A, THCA-B, THCA-C4, THC-C1, THCA-C1, THCV, THCVA, OTHC, CBCF, CBF, cannabiglendol, CBR, cannbicitran, DCBF, cis-THC, triOH-THC, OH-iso-HHCV, synthetic cannabinoids, and/or combinations of any of the foregoing. For example, in some embodiments, the nanoparticle composition may comprise CBN, CBD, and CBG. In some embodiments, the nanoparticle does not contain a CBC, CBCA, CBCV, CBCVA, CBL, CBLA, CBLV, CBD, CBDM, CBDA, CBD-C1, CBDV, CBDVA, CBEA-B, CBE, CBEA-A, CBG, CBGM, CBGA, CBGAM, CBGV, CBGVA, CBND, CBVD, CBN, CBNM, CBN-C2, CBN-C4, CBNA, CBN-C1, CBV, 10-cthoxy-9-hydroxy-delta-6a-tetrahydrocannabinol, 8,9-dihydroxy-delta-61-tetrahydrocannabinol, CBT, CBTV, THC, THC-C4, THCA-A, THCA-B, THCA-C4, THC-C1, THCA-C1, THCV, THCVA, OTHC, CBCF, CBF, cannabiglendol, CBR, cannbicitran, DCBF, cis-THC, triOH-THC, OH-iso-HHCV, and/or a synthetic cannabinoid.

In several embodiments, a plant extract, compound isolated from a plant extract, and/or an active agent may be provided in a salt form. In several embodiments, salt is a pharmaceutically acceptable salt. In several embodiments, the salt is the acetate or citrate salt. In several embodiments, the composition may comprise mixtures of salt forms.

In several embodiments, when formulated, the dry weight % of one or more active agents (e.g., pharmaceuticals, nutraceuticals, cosmetics, pigments, flavorings, etc.) present in the nanoparticle compositions is equal to, between, at most, or at least about: 0.002%, 0.005%, 0.01%, 0.05%, 0.1%, 0.5%, 1%, 5%, 7.5%, 10%, 12.5%, 15%, 20%, 25%, 30%, 35%, 40%, 45%, 50%, or ranges including and/or spanning the aforementioned values. In several embodiments, the active agents are provided in an aqueous composition. In several embodiments, the wet weight % of one or more active agents (e.g., pharmaceuticals, nutraceuticals, cosmetics, pigments, flavorings, etc.) present in the composition (with water included) is equal to, between, at most, or at least about: 0.001%, 0.005%, 0.01%, 0.05%, 0.1%, 0.5%, 1%, 2%, 3%, 4%, 5%, 7.5%, 10%, 12.5%, 15%, 17.5%, 20%, or ranges including and/or spanning the aforementioned values. In several embodiments, the one or more active agents (e.g., pharmaceuticals, nutraceuticals, cosmetics, pigments, flavorings, etc.) may be provided in the wet composition at a concentration of greater than, less than, between, or equal to about: 0.01 mg/mL, 0.05 mg/mL, 0.1 mg/mL, 0.5 mg/mL, 1 mg/mL, 5 mg/mL, 20 mg/mL, 30 mg/mL, 50 mg/mL, 100 mg/mL, 150 mg/mL or ranges including and/or spanning the aforementioned values.

In several embodiments, as disclosed elsewhere herein, the composition is aqueous, while in others it has been dried into a powder (that is free of or substantially free of water). In several embodiments, where the composition has been dried, it comprises a water content of less than or equal to 20%, 15%, 10%, 7.5%, 5%, 2.5%, 1%, or ranges including and/or spanning the aforementioned values.

In several embodiments, as disclosed elsewhere herein, the nanoparticle (or compositions comprising the nanoparticle) may be used to deliver a combination of active ingredients (e.g., 1, 2, 3, 4, or more). In several embodiments, the composition comprises combinations of active compounds of varying ratios. For example, a first active compound to a second active compound present in the composition may be about: 100:1, 90:1, 80:1, 70:1, 60:1, 50:1, 40:1, 30:1, 20:1, 10:1, 5:1, 4:1, 3:1 2:1, 1:1, 1:2, 1:3, 1:4, 1:5, 1:10, 1:20, 1:30, 1:40, 1:50, 1:60, 1:70, 1:80, 1:90, 1:100, or ratios including and/or spanning the aforementioned ratios.

In several embodiments, as disclosed elsewhere herein, the composition is aqueous (e.g., contains water) while in other embodiments, the composition is dry (lacks water or substantially lacks water). In several embodiments, the composition has been dried (e.g., has been subjected to a process to remove most or substantially all water). In several embodiments, the composition comprises nanoparticles in water (e.g., as a solution, suspension, or emulsion). In other embodiments, the composition is provided as a powder (e.g., that may be constituted or reconstituted in water). In several embodiments, as disclosed elsewhere herein, the water content (in wt. %) of the composition is less than or equal to about: 30%, 20%, 10%, 5%, 2.5%, 1%, 0.5%, 0.1%, 0%, or ranges including and/or spanning the aforementioned values. In several embodiments, as disclosed elsewhere herein, the water content (in wt. %) of the composition is greater than or equal to about: 50%, 60%, 70%, 80%, 85%, 90%, 92.5%, 95%, 97.5%, or ranges including and/or spanning the aforementioned values. In several embodiments, the water is nanopure, deionized, USP grade, WFI, and/or combinations of the foregoing. In some aspects, the composition is a dried composition comprising a nanoparticle having weight ratios of a first therapeutic active agent: a lipid source: and optionally a surfactant of 0.002 to 50:2 to 87.5:0 to 17.5.

In several embodiments, as disclosed elsewhere herein, the nanoparticle composition provides an oil-in-water emulsion (e.g., a nanoemulsion), water-in-oil emulsion, a water-in-oil-in-water emulsion, an oil-in-water-in-oil emulsion, a liposome (and variants including multi-lamellar, double liposome preparations, etc.), micelle, and/or solid lipid particles. Any one of these structures may be provided as a nanoparticle or microparticle.

As disclosed elsewhere herein, in some embodiments, the nanoparticle composition comprises a lipid source. In several embodiments, the lipid source comprises a charged lipid, which can impart a charge to the nanoparticle. In several embodiments, the lipid source comprises a neutral lipid. In several embodiments, the lipid source comprises one or more phospholipids. In several embodiments, the one or more phospholipids comprises one or more of phosphatidic acid, phosphatidylethanolamine, phosphatidylcholine, phosphatidylserine, phosphatidylinositol, phosphatidylinositol phosphate, phosphatidylinositol bisphosphate, phosphatidylinositol trisphosphate, lipoid H 100-3, phospholipon 90H, phospholipon 80H, lipoid 100-3, lipoid P75-3, or any combination of the foregoing. In several embodiments, the lipid source is a phosphatidylcholine. In several embodiments, the only lipid present is a phosphatidylcholine (e.g., the lipid source lacks phospholipids other than phosphatidylcholine or is substantially free of other phospholipids). In several embodiments, the one or more lipid source lipid(s) (collectively or individually) are present in the composition at a dry wt. % of equal to or less than about: 0%, 5%, 10%, 15%, 20%, 30%, 40%, 50%, 60%, 70%, 80%, 90%, or ranges including and/or spanning the aforementioned values. In several embodiments, the one or more lipid source lipid(s) (collectively or individually) are present in the composition at a wet wt. % of equal to or less than about: 0%, 0.1%, 0.5%, 1.0%, 2.5%, 4%, 5%, 6%, 7.5%, 10%, 12.5%, 15%, 17.5%, 20%, 25%, 30%, 35%, 40%, 45%, 50%, or ranges including and/or spanning the aforementioned values. In several embodiments, the one or more lipid source lipid(s) (collectively or individually) are present in the composition at a wet w/v of equal to or less than about: 0 mg/mL, 0.1 mg/mL, 0.5 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, 20 mg/mL, 25 mg/mL, 30 mg/mL, 35 mg/mL, 40 mg/mL, 45 mg/mL, 50 mg/mL, 100 mg/mL, 150 mg/mL, 200 mg/mL, 250 mg/mL, 300 mg/mL, 350 mg/mL, 400 mg/mL, 450 mg/mL, 500 mg/mL or ranges including and/or spanning the aforementioned values. In several embodiments, as disclosed elsewhere herein, the composition is aqueous, while in others it has been dried into a powder. For instance, as disclosed elsewhere herein, in several embodiments, the composition is aqueous (wet), while in others it has been dried into a powder (dry). In several embodiments, the one or more lipid(s) of the lipid source are synthetic, derived from sunflower, soy, egg, or mixtures thereof. In several embodiments, the one or more lipids of the lipid source can be hydrogenated or non-hydrogenated. In several embodiments, the lipid source exceeds requirements of the United States Pharmacopeia (is USP grade) and/or is National Formulary (NF) grade.

In several embodiments, the lipid source (e.g., phosphatidylcholine, including hydrogenated soybean phosphatidylcholine) may be of high purity. For example, in some embodiments, the phosphatidylcholine is H100-3 grade (from Lipoid) and includes over 96.3% phosphatidylcholine (hydrogenated) or over 99% phosphatidylcholine (hydrogenated). In several embodiments, the one or more lipids of the lipid source has a purity of greater than or equal to about: 92.5%, 95%, 96%, 96.3%, 98%, 99%, 100%, or ranges including and/or spanning the aforementioned values. In several embodiments, the one or more lipids of the lipid source has a total % impurity content by weight of less than or equal to about: 8.5%, 5%, 4%, 3.7%, 2%, 1%, 0%, or ranges including and/or spanning the aforementioned values. In several embodiments, the one or more lipids of the lipid source comprises less than or equal to about 8.5%, 5%, 4%, 3.7%, 2%, 1%, or 0.1% (or ranges including and/or spanning the aforementioned values) of any one or more of saturated fatty acids, monounsaturated fatty acids, polyunsaturated fatty acids (C 18), arachidonic acid (ARA) (C 20:4), docosahexaenoic acid DHA (C 22:6), phosphatidic acid, phosphatidylethanolamine, and/or lysophosphatidylcholine by weight. In several embodiments, the one or more lipids of the lipid source has less than about 1.1% lysophosphatidylcholine and less than about 2.0% triglycerides by weight.

In some embodiments, the lipid source (e.g., phosphatidylcholine, including hydrogenated soybean phosphatidylcholine) may be 90%, 80%, 70%, 60%, 50%, 40%, 30%, 20% pure, or ranges including and/or spanning the aforementioned values.

As disclosed elsewhere herein, in some embodiments, the nanoparticle composition comprises a surfactant. In some embodiments, the nanoparticle composition does not comprises a surfactant. In several embodiments, the surfactant is a pharmaceutically acceptable surfactant. In several embodiments, the surfactant is a food surfactant. In several embodiments, the surfactant comprises one or more of a polyoxyethylene sorbitan esters (e.g., polysorbates/tweens, including polysorbate 80, polysorbate 20, etc.), cremophor (e.g., a non-ionic solubilizer and emulsifier that is made by reacting ethylene oxide with castor oil), propylene oxide-modified polymethylsiloxane, dodecyl betaine, lauramidopropyl betaine, cocoamido-2-hydroxypropyl sulfobetaine, sodium stearate (or other stearate salts), polyoxyethylene alcohol, lecithins, mono- and diglycerides of fatty acids (MDG), acetic acid esters of MDG, lactic acid esters of MDG, citric acid esters of MDG, mono- and diacetyl tartaric acid esters of MDG, sucrose esters of fatty acids, polyglycerol esters of fatty acids (e.g., polyglycerol esters), polyglycerol polyricinoleate, propane-1,2-diol esters of fatty acids, propylene glycol esters, sodium stearoyl-2-lactylate, calcium stearoyl-2-lactylate, sorbitan fatty acid esters, quillaja extract surfactant, yucca extract surfactant, saponins, silicone emulsifiers, sorbitan trioleate, soya lecithin, dioctyl sodium sulfosuccinate, dioctyl sodium sulfonate, polyoxyethylene, hydrogenated castor oil, sucrose fatty acid ester, or combinations of any of the foregoing. Natural or synthetic surfactants can be used, including polyethylene glycol and dextrans, such as cyclodextran. In several embodiments, the one or more surfactants are present in the nanoparticle composition (collectively or individually) at a dry wt. % of equal to or less than about: 0%, 5%, 10%, 15%, 20%, 30%, 40%, 50%, 60%, or ranges including and/or spanning the aforementioned values. Surfactants can include cationic, anionic, non-ionic, and zwitterionic surfactants. In several embodiments, the one or more surfactants (collectively or individually) are present in the composition at a wet wt. % of equal to or less than about: 0%, 0.1%, 0.5%, 1.0%, 2.5%, 4%, 5%, 6%, 7.5%, 10%, 12.5%, 15%, 17.5%, or ranges including and/or spanning the aforementioned values. In several embodiments, the one or more surfactants (collectively or individually) are present in the composition at a wet w/v of equal to or less than about: 0 mg/mL, 0.1 mg/mL, 0.5 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, or ranges including and/or spanning the aforementioned values. In several embodiments, the surfactant exceeds requirements of the United States Pharmacopeia (is USP grade) and/or is National Formulary (NF) grade.

In several embodiments, one or more co-emulsifiers are used. In several embodiments, the co-emulsifier is a pharmaceutically acceptable co-emulsifier. In several embodiments, the co-emulsifier is selected from the group consisting of oleic acid, miglyol 812N (all versions), triglycerides, conjugated linoleic acid (CLA), cetearyl olivate, isoprpyle myristate, glyceryl stearate (e.g., glycerol monostearate), celluloses and polysaccharides (e.g., methylcellulose, propylmethylcellulose, hydroxypropyl methylcellulose, xanthan gum, etc.) and/or combinations of any of the foregoing. In several embodiments, the one or more co-emulsifiers are present in the nanoparticle composition (collectively or individually) at a dry wt. % of equal to or less than about: 0%, 0.01%, 0.05%, 0.1%, 0.2%, 0.3%, 0.4%, 0.5%, 0.6%, 0.7%, 0.8%, 0.9%, 1%, 2%, 3%, 4%, 5%, 10%, 15%, 20%, 30%, 40%, 50%, 60%, or ranges including and/or spanning the aforementioned values. In several embodiments, the one or more co-emulsifiers (collectively or individually) are present in the composition at a wet wt. % of equal to or less than about: 0%, 0.01%, 0.05%, 0.1%, 0.2%, 0.3%, 0.4%, 0.5%, 0.6%, 0.7%, 0.8%, 0.9%, 1.0%, 2.5%, 4%, 5%, 6%, 7.5%, 10%, 12.5%, 15%, 17.5%, or ranges including and/or spanning the aforementioned values. In several embodiments, the one or more co-emulsifiers (collectively or individually) are present in the composition at a wet w/v of equal to or less than about: 0 mg/mL, 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 2.5 mg/mL, 4 mg/mL, 5 mg/mL, 6 mg/mL, 7.5 mg/mL, 10 mg/mL, 12.5 mg/mL, 15 mg/mL, 17.5 mg/mL, or ranges including and/or spanning the aforementioned values. In several embodiments, the co-emulsifiers exceeds requirements of the United States Pharmacopcia (is USP grade) and/or is National Formulary (NF) grade.

In some embodiments, the co-emulsifier component comprises a medium chain triglyceride (MCT) or a MCT-substitute. In some embodiments, the medium chain triglyceride comprises a fatty acid selected from one or more of caproic acid, octanoic acid, capric acid, caprylic acid, and/or lauric acid (e.g., is formed from). In some embodiments, the medium chain triglyceride comprises a fatty acid 6-12 carbons in length (e.g., 6, 7, 8, 9, 10, 11, or 12). In some embodiments, the co-emulsifier component comprises a long chain triglyceride (LCT). In some embodiments, the long chain triglyceride comprises a fatty acid greater than 12 carbons in length (e.g., greater than or equal to 13, 14, 15, 16, 17, 18, 19, or 20 carbons in length, or ranges including and/or spanning the aforementioned values). In some embodiments, the co-emulsifier component is a single lipid. In some embodiments, the co-emulsifier component is MCT. In some embodiments, the MCT is highly pure. In some embodiments, the MCT has a purity by weight % of equal to or greater than about: 90%, 95%, 97%, 98%, 99%, 100%, or ranges including and/or spanning the aforementioned values. In some embodiments, the MCT (or LCT) is present in the nanoparticle composition at dry weight % of equal to or greater than about: 10%, 20%, 30%, 35%, 40%, 45%, 50%, or ranges including and/or spanning the aforementioned values. In some embodiments, the MCT-substitute lipid (e.g., the non-phospholipid lipid) is selected from one or more of oleic acid, capric acid, caprylic acid, and triglycerides of such (Captex 8000, Captex GTO, Captex 1000), glycerol monooleate, glycerol monostearate (Geleol™ Mono and Diglyceride NF), omega-3 fatty acids (α-linolenic acid (ALA), cicosapentaenoic acid (EPA), docosahexaenoic acid (DHA), Tonalin, Pronova Pure® 46:38, free fatty acid Tonalin FFA 80), conjugated linoleic acid, alpha glycerylphosphorylcholine (alpha GPC), palmitoylethanolamide (PEA), cetyl alcohol, or emulsifying wax. In some embodiments, the one or more MCT-substitute(s) are present in the nanoparticle composition (collectively or individually) at a dry wt. % of equal to or less than about: 0.5%, 1.0%, 2.5%, 5%, 10%, 15%, 20%, 30%, 40%, 50%, 60%, 80% or ranges including and/or spanning the aforementioned values. In some embodiments, the one or more MCT-substitute(s) (collectively or individually) are present in the composition at a wet wt. % of equal to or less than about: 0.5%, 1.0% 2.5%, 5%, 7.5%, 10%, 12.5%, 15%, 20%, 30%, 40%, 60% or ranges including and/or spanning the aforementioned values. In some embodiments, the MCT-substitute has a purity of greater than or equal to about: 70%, 80%, 85%, 92.5%, 95%, 96%, 98%, 99%, 99.9%, 100%, or ranges including and/or spanning the aforementioned values. In some embodiments, the MCT-substitute has a total % impurity content by weight of less than or equal to about: 8.5%, 5%, 4%, 3.7%, 2%, 1%, 0%, or ranges including and/or spanning the aforementioned values.

In some embodiments, the co-emulsifier component comprises one or more gums. In some embodiments, the gum is a xanthan gum and/or a biosaccharide gum, such as biosaccharide gum-1,-2,-3,-4,-5, etc. In some embodiments, the gum provides increased stability of the nanoparticle over time and/or at higher temperatures. Higher temperatures may include exposure of the nanoparticles to a hot liquid, such as a hot aqueous liquid such as a hot beverage (e.g., coffee, tea, hot chocolate, etc.). The higher temperatures may be temperatures such as 70° C. or higher. Higher temperatures may include above, at, below, between, or any range of 70° C. to 100° C., such as 70° C. to 85° C., 70° C. to 80° C., 70° C., 75° C., 80° C., 85° C., 95° C., or 100° C. In several embodiments, after a 30 minute period in a hot liquid, the particle size and/or PDI varies by less than or equal to about: 1%, 5%, 10%, 20%, 30%, or ranges including and/or spanning the aforementioned values. In several embodiments, after a 30 minute period in a hot liquid, the active agent (e.g., pharmaceuticals, nutraceuticals, cosmetics, pigments, flavorings, and the like) concentration drops by less than or equal to about: 1%, 5%, 10%, 15%, or ranges including and/or spanning the aforementioned values. In several embodiments, the one or more gums are present in the nanoparticle composition (collectively or individually) at a dry wt. % of equal to or less than about: 0%, 0.01%, 0.05%, 0.1%, 0.2%, 0.3%, 0.4%, 0.5%, 0.6%, 0.7%, 0.8%, 0.9%, 1%, 2%, 3%, 4%, 5%, or ranges including and/or spanning the aforementioned values. In several embodiments, the one or more gums (collectively or individually) are present in the composition at a wet wt. % of equal to or less than about: 0%, 0.01%, 0.05%, 0.1%, 0.2%, 0.3%, 0.4%, 0.5%, 0.6%, 0.7%, 0.8%, 0.9%, 1.0%, 2.5%, 4%, 5%, or ranges including and/or spanning the aforementioned values. In several embodiments, the one or more gums (collectively or individually) are present in the composition at a wet w/v of equal to or less than about: 0 mg/mL, 0.01 mg/mL, 0.05 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 2.5 mg/mL, 4 mg/mL, 5 mg/mL, or ranges including and/or spanning the aforementioned values.

In some embodiments, the nanoparticle composition comprises one or more sterols. In some embodiments, the one or more sterols comprises one or more cholesterols, ergosterols, hopanoids, hydroxysteroids, phytosterols (e.g., VEGAPURE®), ecdysteroids, and/or steroids. In some embodiments the sterol comprises a cholesterol. In some embodiments, the sterol component is a single sterol. In some embodiments, the sterol component is cholesterol. In some embodiments, the cholesterol (or other sterol) is highly pure. In some embodiments, the one or more sterol(s) (e.g., cholesterol, and/or other sterols), collectively or individually, are present in the aqueous composition at a concentration of less than or equal to about: 50 mg/mL, 40 mg/mL, 20 mg/mL, 10 mg/mL, 5 mg/mL, or ranges including and/or spanning the aforementioned values. In some embodiments, the one or more sterol(s) are present in the composition at a dry wt. % of equal to or less than about: 0.25%, 0.5%, 1%, 5%, 7.5%, 10%, 15%, 20%, 25%, or ranges including and/or spanning the aforementioned values. In some embodiments, the one or more sterol(s) (collectively or individually) are present in the composition at a wet wt. % of equal to or less than about: 0.1%, 0.25%, 0.5%, 1%, 2%, 3%, 4%, 5%, 7.5%, 10%, or ranges including and/or spanning the aforementioned values. In some embodiments, the cholesterol used in the composition comprises cholesterol from one or more of sheep's wool, synthetic cholesterol, or semisynthetic cholesterol from plant origin. In some embodiments, the sterol has a purity of greater than or equal to about: 92.5%, 95%, 96%, 98%, 99%, 99.9%, 100.0%, or ranges including and/or spanning the aforementioned values. In some embodiments, the sterol has a total % impurity content by weight of less than or equal to about: 8.5%, 5%, 4%, 3.7%, 2%, 1%, 0%, or ranges including and/or spanning the aforementioned values. In some embodiments, the sterol is cholesterol. In some embodiments, the sterol is not cholesterol. In some embodiments, the sterol is a phytosterol.

In several embodiments, the nanoparticle composition comprises a preservative. In several embodiments, the preservative includes one or more benzoates (such as sodium benzoate or potassium benzoate), nitrites (such as sodium nitrite), sulfites (such as sulfur dioxide, sodium or potassium sulphite, bisulphite or metabisulphite), sorbates (such as sodium sorbate, potassium sorbate), ethylenediaminetetraacetic acid (EDTA) (and/or the disodium salt thereof), polyphosphates, organic acids (e.g., citric, succinic, ascorbic, malic, tartaric, benzoic, lactic and propionic acids), extracts (e.g., mushroom extract) and/or antioxidants (e.g., vitamins such as vitamin E and/or vitamin C, butylated hydroxytoluene). In several embodiments, sorbates and benzoates may be used in acidic pH formulations. In several embodiments, the one or more preservatives (collectively or individually) are present in the composition at a dry wt. % of equal to or at less than about: 0.01%, 0.1%, 0.25%, 0.5%, 1%, 5%, 7.5%, 10%, 15%, 20%, 25%, or ranges including and/or spanning the aforementioned values. In several embodiments, the one or more preservatives (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5%, or ranges including and/or spanning the aforementioned values. In several embodiments, the one or more surfactants (collectively or individually) are present in the composition at a wet w/v of equal to or less than about: 0 mg/mL, 0.001 mg/mL, 0.1 mg/mL, 0.5 mg/mL, 1.0 mg/mL, 2.5 mg/mL, 4 mg/mL, 5 mg/mL, or ranges including and/or spanning the aforementioned values. In several embodiments, as disclosed elsewhere herein, the composition is aqueous, while in others it has been dried into a powder. For instance, as disclosed elsewhere herein, in several embodiments, the composition is aqueous (wet), while in others it has been dried into a powder (dry). In several embodiments, the preservatives inhibit or prevent growth of mold, bacteria, and fungus.

In several embodiments, the nanoparticle composition comprises one or more flavoring agents. In several embodiments, the one or more flavoring agent(s) comprise an essential oil (or combinations of essential oils). In several embodiments, the one or more flavoring agents of the composition comprise monk fruit extract (e.g., MonkGold50), stevia, glycerin, peppermint oil or flavoring, lemon oil or flavoring, orange oil or flavoring, vanilla, taste makers, bitter blockers, or the like, or combinations thereof. In several embodiments, the one or more flavoring agent(s) (collectively or individually) are present in the composition at a dry wt. % of equal to or less than about: 0.01%, 0.1%, 0.25%, 0.5%, 1%, 5%, 7.5%, 10%, 15%, 20%, 25%, or ranges including and/or spanning the aforementioned values. In several embodiments, the one or more flavoring agents (collectively or individually) are present in the composition at a wet wt. % of equal to or less than about: 0.001%, 0.01%, 0.025%, 0.05%, 0.1%, 0.5%, 0.75%, 1.0%, 1.5%, 2.0%, 2.5%, 5.0%, or ranges including and/or spanning the aforementioned values. In several embodiments, as disclosed elsewhere herein, the composition is aqueous, while in others it has been dried into a powder. For instance, as disclosed elsewhere herein, in several embodiments, the composition is aqueous (wet), while in others it has been dried into a powder (dry).

In several embodiments, the composition comprises a combination of lipid sources and a sterol. In several embodiments, the composition comprises an active agent or combination of actives and a lipid source. In several embodiments, as disclosed elsewhere herein, the nanoparticle composition comprises an active agent or combination of actives and a surfactant. In several embodiments, as disclosed elsewhere herein, the nanoparticle composition comprises an active agent or combination of actives and a co-emulsifier (e.g., oleic acid, miglyol 812N (all versions), triglycerides, conjugated linoleic acid (CLA), cetearyl olivate, isoprpyle myristate, glyceryl stearate, etc.). In several embodiments, the composition further comprises a flavoring agent. In several embodiments, the composition further comprises a preservative.

In several embodiments, the composition comprises, consists of, or consists essentially of one or more active agents, one or more lipid sources, one or more surfactants, one or more flavoring agents, one or more preservatives, one or more co-emulsifiers, or any combination thereof. In several embodiments, the composition comprises, consists of, or consists essentially of one or more active agents, one or more lipid sources, one or more surfactants, one or more preservatives, and one or more co-emulsifiers. In several embodiments, the composition comprises, consists of, or consists essentially of one or more active agents, one or more lipid sources, one or more surfactants, one or more flavoring agents, and one or more co-emulsifiers. In several embodiments, the composition comprises, consists of, or consists essentially of one or more active agents, one or more surfactants, and one or more co-emulsifiers. In several embodiments, the composition comprises, consists of, or consists essentially of one or more active agents, one or more lipid sources, one or more surfactants, one or more flavoring agents, and one or more preservatives. In several embodiments, the composition comprises, consists of, or consists essentially of one or more active agents, one or more lipid sources, and one or more surfactants. In several embodiments, the composition comprises, consists of, or consists essentially of one or more active agents, one or more lipid sources, one or more surfactants, and one or more preservatives. In several embodiments, the composition comprises, consists of, or consists essentially of one or more active agents, one or more surfactants, one or more flavoring agents, and one or more preservatives. In several embodiments, the compositions above do not comprise a surfactant. In several embodiments, the compositions above do not comprise an active agent.

In several embodiments, the nanoparticle composition lacks terpenes (e.g., as impurities or additives). However, in other embodiments, one or more terpenes may be added to prepare the nanoparticle composition. In several embodiments, the one or more terpenes includes one or more of alpha fenchone, alpha terpinene, alpha terpincol, beta caryophyllene, alpha pinene, beta pinene, bisabolene, bisabolol, borneol, cucalyptol, gamma terpinene, guaiacol, humulene, linalool, myrcene, para cymene, phytol, and/or terpinolene. In several embodiments, the one or more terpenes includes one or more of 7,8-dihydro-alpha-ionone, 7,8-dihydro-beta-ionone, Acctanisole, Acetic Acid, Acetyl Cedrene, Ancthole, Anisole, Benzaldehyde, Bergamotene (Alpha-cis-Bergamotene) (Alpha-trans-Bergamotene), Bisabolol (Beta-Bisabolol), Alpha Bisabolol, Borncol, Bornyl Acetate, Butanoic/Butyric Acid, Cadinenc (Alpha-Cadinene) (Gamma-Cadinenc), Cafestol, Caffeic acid, Camphenc, Camphor, Capsaicin, Carene (Delta-3-Carenc), Carotene, Carvacrol, Dextro-Carvone, Laevo-Carvonc, Alpha-Caryophyllene, Beta-Caryophyllene, Caryophyllene oxide, Cedrene (Alpha-Cedrene) (Beta-Cedrene), Cedrene Epoxide (Alpha-Cedrene Epoxide), Cedrol, Cembrene, Chlorogenic Acid, Cinnamaldehyde, Alpha-amyl-Cinnamaldehyde, Alpha-hexyl-Cinnamaldehyde, Cinnamic Acid, Cinnamyl Alcohol, Citronellal, Citronellol, Cryptone, Curcumene (Alpha-Curcumene) (Gamma-Curcumene), Decanal, Dehydrovomifoliol, Diallyl Disulfide, Dihydroactinidiolide, Dimethyl Disulfide, Eicosane/Icosane, Elemene (Beta-Elemenc), Estragole, Ethyl acetate, Ethyl Cinnamate, Ethyl maltol, Eucalyptol/1,8-Cineole, Eudesmol (Alpha-Eudesmol) (Beta-Eudesmol) (Gamma-Eudesmol), Eugenol, Euphol, Farnesene, Farnesol, Fenchol (Beta-Fenchol), Fenchone, Geraniol, Geranyl acetate, Germacrenes, Germacrene B, Guaia-1(10),11-diene, Guaiacol, Guaiene (Alpha-Guaiene), Gurjunene (Alpha-Gurjunene), Herniarin, Hexanaldehyde, Hexanoic Acid, Humulene (Alpha-Humulene) (Beta-Humulene), Ionol (3-oxo-alpha-ionol) (Beta-Ionol), Ionone (Alpha-Ionone) (Beta-Ionone), Ipsdienol, Isoamyl Acetate, Isoamyl Alcohol, Isoamyl Formate, Isoborneol, Isomyrcenol, Isopulegol, Isovaleric Acid, Isoprene, Kahweol, Lavandulol, Limonene, Gamma-Linolenic Acid, Linalool, Longifolene, Alpha-Longipinene, Lycopene, Menthol, Methyl butyrate, 3-Mercapto-2-Methylpentanal, Mercaptan/Thiols, Beta-Mercaptoethanol, Mercaptoacetic Acid, Allyl Mercaptan, Benzyl Mercaptan, Butyl Mercaptan, Ethyl Mercaptan, Methyl Mercaptan, Furfuryl Mercaptan, Ethylene Mercaptan, Propyl Mercaptan, Thenyl Mercaptan, Methyl Salicylate, Methylbutenol, Methyl-2-Methylvalerate, Methyl Thiobutyrate, Myrcene (Beta-Myrcene), Gamma-Muurolene, Nepetalactone, Nerol, Nerolidol, Neryl acetate, Nonanaldehyde, Nonanoic Acid, Ocimene, Octanal, Octanoic Acid, P-Cymene, Pentyl butyrate, Phellandrene, Phenylacetaldehyde, Phenylethanethiol, Phenylacetic Acid, Phytol, Pinene, Beta-Pinene, Propanethiol, Pristimerin, Pulegone, Quercetin, Retinol, Rutin, Sabinene, Sabinene Hydrate, cis-Sabinene Hydrate, trans-Sabinene Hydrate, Safranal, Alpha-Selinene, Alpha-Sinensal, Beta-Sinensal, Beta-Sitosterol, Squalene, Taxadiene, Terpin hydrate, Terpineol, Terpine-4-ol, Alpha-Terpinene, Gamma-Terpinene, Terpinolene, Thiophenol, Thujone, Thymol, Alpha-Tocopherol, Tonka Undecanone, Undecanal, Valeraldehyde/Pentanal, Verdoxan, Alpha-Ylangene, Umbelliferone, or Vanillin.

In several embodiments, the composition may also comprise one or more terpenes. In several embodiments, the one or more terpenes, collectively or individually, are present in the aqueous composition at a concentration of less than or equal to about: 400 mg/mL, 300 mg/mL, 200 mg/mL, 150 mg/mL, 100 mg/mL, 75 mg/mL, 50 mg/mL, 25 mg/mL, or ranges including and/or spanning the aforementioned values. In several embodiments, the one or more terpenes (collectively or individually) are present in the composition at a dry wt. % of equal to or less than about: 5%, 10%, 15%, 20%, 30%, 40%, 50%, 60%, or ranges including and/or spanning the aforementioned values. In several embodiments, the one or more terpenes (collectively or individually) are present in the composition at a wet wt. % of equal to or less than about: 2.5%, 5%, 7.5%, 10%, 12.5%, 15%, 20%, 30%, 40%, or ranges including and/or spanning the aforementioned values.

In several embodiments, as disclosed elsewhere herein, the nanoparticle composition provides particles in the nano-measurement range. In several embodiments, the nanoparticle is spherical or substantially spherical. In several embodiments, a solid lipid nanoparticle possesses a solid lipid core matrix that can solubilize lipophilic molecules. In several embodiments, the lipid core is stabilized by surfactants and/or emulsifiers as disclosed elsewhere herein, while in other embodiments, surfactants are absent. In several embodiments, the size of the particle is measured as a mean diameter. In several embodiments, the size of the particle is measured by dynamic light scattering. In several embodiments, the size of the particle is measured using a zeta-sizer. In several embodiments, the size of the particle can be measured using Scanning Electron Microscopy (SEM). In several embodiments, the size of the particle is measured using a cyrogenic SEM (cryo-SEM). Where the size of a nanoparticle is disclosed elsewhere herein, any one or more of these instruments or methods may be used to measure such sizes.

In several embodiments, the nanoparticle composition comprises nanoparticles having an average size of less than, greater than, between, or equal to about: 10 nm, 25 nm, 40 nm, 50 nm, 100 nm, 250 nm, 500 nm, 1000 nm, or ranges including and/or spanning the aforementioned values. In several embodiments, the composition comprises nanoparticles having an average size of between about 50 nm and 150 nm or between about 50 and about 250 nm. In several embodiments, the size distribution of the nanoparticles for at least 50%, 75%, 80%, 90% (or ranges including and/or spanning the aforementioned percentages) of the particles present is equal to or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In several embodiments, the size distribution of the nanoparticles for at least 90% of the particles present is equal to, between, greater than, or less than about: 20 nm, 40 nm, 60 nm, 80 nm, 100 nm, 110 nm, 120 nm, 130 nm, 140 nm, 160 nm, 180 nm, 200 nm, 300 nm, 400 nm, 500 nm, or ranges including and/or spanning the aforementioned nm values. In several embodiments, the D90 of the particles present is equal to, between, greater than, or less than about: 80 nm, 100 nm, 110 nm, 120 nm, 130 nm, 140 nm, 160 nm, 180 nm, 200 nm, 300 nm, 400 nm, 500 nm, or ranges including and/or spanning the aforementioned values. In several embodiments, the size of the nanoparticle is the diameter of the nanoparticle as measured using any of the techniques as disclosed elsewhere herein. For instance, in some embodiments, the size of the nanoparticle is the measured using dynamic light scattering. In several embodiments, the size of the nanoparticle is the measured using a zeta sizer. In several embodiments, consistency in size over time, or within a sample, allows predictable stability for the active agent encapsulated therein.

In several embodiments, over 50%, 75%, 95% (or ranges spanning and or including the aforementioned values) of the nanoparticles prepared by the methods disclosed herein have a particle size of between about 20 to about 500 nm (as measured by zeta sizing (e.g., refractive index). In several embodiments, over 50%, 75%, 95% (or ranges spanning and or including the aforementioned values) of the nanoparticles prepared by the methods disclosed herein have a particle size of between about 50 nm to about 200 nm (as measured by zeta sizing (e.g., refractive index). In several embodiments, over 50%, 75%, 95% (or ranges spanning and or including the aforementioned values) of the nanoparticles prepared by the methods disclosed herein have a particle size of between about 90 nm to about 150 nm (as measured by zeta sizing (e.g., refractive index). In several embodiments, this consistency in size allows predictable delivery to subjects. In several embodiments, the D90 particle size measurement varies between 150 and 500 nm.

In several embodiments, as disclosed elsewhere herein, the nanoparticle composition is an oil-in-water emulsion, water-in-oil emulsion, water-in-oil-in-water emulsion, oil-in-water-in-oil emulsion, liposome, solid lipid particles formulation, etc. For brevity, these may just be referred to as the composition. In several embodiments, the nanoparticle composition can be processed to comprises one or more of solid lipid nanoparticles, liposomes (and variants including multi-lamellar, double liposome preparations, etc.), niosomes, ethosomes, electrostatic particulates, microemulsions, nanoemulsions, microsuspensions, nanosuspensions, or combinations thereof. In several embodiments, polymeric nanoparticles may be formed. In several embodiments, cyclodextrin is added.

In several embodiments, as disclosed elsewhere herein, the nanoparticle composition is a dry or substantially dry composition. In some instances, the dry or substantially dry composition is a powder, a pellet, a plurality of particles, a film, a pill, a tablet, etc. In some instances, the dry or substantially dry composition is an oily liquid.

In some instances, composition comprises a carrier, such as a pharmaceutically acceptable carrier. The carrier can be, but is not limited to distilled water, physiological phosphate-buffered saline, normal or lactated Ringer's solutions, dextrose solution, Hank's solution, propanediol, a carbohydrate. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve. The carrier can be a solid carrier, such as a carbohydrate, clay, a salt, an inert powder, a polymer, chalk, etc.

In several embodiments, solid lipid nanoparticle compositions comprises a lipid core matrix. In several embodiments, the lipid core matrix is solid. In several embodiments, the solid lipid comprises one or more ingredients as disclosed elsewhere herein. In several embodiments, the core of the solid lipid comprises one or more lipids, surfactants, active ingredients, etc. In several embodiments, the surfactant acts as an emulsifier. In several embodiments, emulsifiers can be used to stabilize the lipid dispersion (with respect to charge and molecular weight). In several embodiments, the core ingredients (e.g., the components of the core) are present in the composition (collectively or individually) at a dry wt. % of equal to or less than about: 0.5%, 1.0%, 2.5%, 5%, 10%, 15%, 20%, 30%, 40%, 50%, 60%, 80% or ranges including and/or spanning the aforementioned values. In several embodiments, the core ingredients and/or the emulsifiers (collectively or individually) are present in the composition at a wet wt. % of equal to or less than about: 0.5%, 1.0% 2.5%, 5%, 7.5%, 10%, 12.5%, 15%, 20%, 30%, 40%, 60% or ranges including and/or spanning the aforementioned values.

In several embodiments, the nanoparticle composition (e.g., when in water or dried) comprises multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, and/or combinations thereof. In several embodiments, the composition is characterized by having multiple types of particles (e.g., lamellar, emulsion, irregular, etc.). In other embodiments, a majority of the particles present are emulsion particles. In several embodiments, a majority of the particles present are lamellar (multilamellar and/or unilamellar). In other embodiments, a majority of the particles present are irregular particles. In still other embodiments, a minority of the particles present are emulsion particles. In several embodiments, a minority of the particles present are lamellar (multilamellar and/or unilamellar). In other embodiments, a minority of the particles present are irregular particles.

In several embodiments, at ambient temperature an aqueous nanoparticle composition as disclosed herein has a viscosity (in centipoise (cP)) of equal to or less than about: 1.0, 1.05, 1.1, 1.2, 1.5, 2.0, 5.0, 10.0, 20, 30, 50, 100, or ranges including and/or spanning the aforementioned values. In several embodiments, at about 25° C. or 26° C. and a concentration of 20 mg/mL active agent in water (e.g., the total nanoparticle composition may have a concentration of 50 to 250 mg/mL), the nanoparticle composition has a viscosity (in centipoise (cP)) of equal to or less than about: 1.0, 1.05, 1.1, 1.2, 1.5, 2.0, 5.0, 10.0, 20, 30, 50, 100, or ranges including and/or spanning the aforementioned values. In several embodiments, at about 25° C. and a concentration of 50 mg/mL, 100 mg/mL, 200 mg/mL, or 250 mg/mL, the nanoparticle composition has a viscosity (in cP) of equal to or less than about: 1.0, 1.05, 1.1, 1.2, 1.5, 2.0, 5.0, 10.0, 20, 30, 50, 100, or ranges including and/or spanning the aforementioned values. In several embodiments, the viscosity of the lipid nanoparticle aqueous solution is equal to or less than 5.0 Cp.

In several embodiments, the nanoparticle delivery system described herein offers protection to active compounds against degradation in an aqueous environment for long-term storage. In several embodiments, the composition is well characterized to ensure a consistent product from batch to batch and with long-term stability. In several embodiments, the product stability is routinely tested for appearance, particle size and distribution, zeta potential, residual solvents, heavy metals, active compound concentration, and microbial testing and the values measured using these test methods varies (over a period of at least about 1 month or about 6 months at 25° C. with 60% relative humidity) by less than or equal to about: 1%, 5%, 10%, 20%, 30%, or ranges including and/or spanning the aforementioned values. In several embodiments, the particle size and/or PDI varies over a period of at least about 1 month or about 6 months (at 25° C. with 60% relative humidity) by less than or equal to about: 1%, 5%, 10%, 20%, 30%, or ranges including and/or spanning the aforementioned values. As noted elsewhere herein, PDI and size can be measured using conventional techniques disclosed herein. In several embodiments, the active agent (e.g., pharmaceuticals, nutraceuticals, cosmetics, pigments, flavorings, and the like) concentration varies over a period of at least about 1 month or about 6 months (at 25° C. with 60% relative humidity) by less than or equal to about: 1%, 5%, 10%, 15%, or ranges including and/or spanning the aforementioned values. As noted elsewhere herein, PDI and size can be measured using conventional techniques disclosed herein.

In several embodiments, the formulations and/or compositions disclosed herein are stable during sterilization. In several embodiments, the sterilization may include one or more of ozonation, UV treatment, and/or heat treatment. In several embodiments, the particle size and/or PDI after sterilization (e.g., exposure to techniques that allow sterilization of the composition) varies by less than or equal to about: 1%, 5%, 10%, 20%, 30%, or ranges including and/or spanning the aforementioned values. In several embodiments, the active agent (e.g., pharmaceuticals, nutraceuticals, cosmetics, pigments, flavorings, and the like) concentration after sterilization (e.g., exposure to techniques that allow sterilization of the composition) varies (e.g., drops) by less than or equal to about: 1%, 5%, 10%, 15%, or ranges including and/or spanning the aforementioned values.

In several embodiments, the nanoparticle compositions (including after stabilization) disclosed herein have a shelf life of equal to or greater than 6 months, 12 months, 14 months, 16 months, 18 months, 19 months, or ranges including and/or spanning the aforementioned values. The shelf-life can be determined as the period of time in which there is 95% confidence that at least 50% of the response (active agent(s) concentration or particle size) is within the specification limit. This refers to a 95% confidence interval and when linear regression predicts that at least 50% of the response is within the set specification limit.

In several embodiments, the composition contains preservatives to protect against bacteria, mold, and fungal growth. The product specification is no more than 100 cfu/gram. In several embodiments, over a period of about 1 month, about 6 months, or about 12 months the composition has equal to or not more than: 50 cfu/gram, 10 cfu/gram, 5 cfu/gram, 1 cfu/gram, 0.1 cfu/gram, or ranges including and/or spanning the aforementioned values. In several embodiments, 1 week at 20° C.-25° C. after a 10⁵-10⁷ collony forming units (cfu)/mL challenge with any one of Staphylococcus aureus, Pseudomonas aeruginosa, Escherichia coli, Candida albicans, and Aspergillus brasiliensis the composition has equal to or not more than: 100 cfu/gram, 50 cfu/gram, 25 cfu/gram, 10 cfu/gram, 5 cfu/gram, 1 cfu/gram, 0.1 cfu/gram, or ranges including and/or spanning the aforementioned values. In several embodiments, 1 weck at 20° C.-25° C. after a 10⁵-10⁷ cfu/mL challenge with any one of Staphylococcus aureus, Pseudomonas aeruginosa, Escherichia coli, Candida albicans, and Aspergillus brasiliensis the composition has a log reduction for the bacteria of equal to or greater than: 1, 2, 3, 4, 5, 10, or ranges including and/or spanning the aforementioned values.

The shelf-life can be determined as the period of time in which there is 95% confidence that at least 50% of the response (active agent(s) concentration or particle size) is within a specification limit. A specification limit is a range of measured values in which a quality parameter should be within in order for products to be considered of the same quality when it was initially released. For example, where the active ingredient concentration is 20 mg/mL, a specification limit may be defined as 18 to 22 mg/mL, and during a stability study if the active agent concentration falls below 18 mg/mL due to chemical instability, at that time the product may be considered out of specification.

In several embodiments, the shelf life is determined as a time where the concentration of the active ingredient has changed (e.g., lessened) by less than or equal to 15%, 10%, 5%, 2.5%, or ranges including and or spanning the aforementioned ranges.

In several embodiments, the nanoparticle delivery system described herein offers protection to active compounds against degradation at higher temperatures, such as in hot aqueous environments. Hot aqueous environments can include hot liquids such as a hot beverage. The higher temperatures may be temperatures such as 70° C. or higher. Higher temperatures may include above, at, below, between, or any range of 70° C. to 100° C., such as 70° C. to 85° C., 70° C. to 80° C., 70° C., 75° C., 80° C., 85° C., 95° C., or 100° C. In several embodiments, after a 30 minute period in a hot liquid, the particle size and/or PDI varies by less than or equal to about: 1%, 5%, 10%, 20%, 30%, or ranges including and/or spanning the aforementioned values. In several embodiments, after a 30 minute period in a hot liquid, the active agent (e.g., pharmaceuticals, nutraceuticals, cosmetics, pigments, flavorings, and the like) concentration drops by less than or equal to about: 1%, 5%, 10%, 15%, or ranges including and/or spanning the aforementioned values.

In some embodiments, the density of the composition is purposefully modified. In some embodiments, the density is approximately 0.7 g/mL, 0.75 g/mL, 0.8 g/mL, 0.85 g/mL, 0.9 g/mL, 0.95 g/mL, 1.0 g/mL, 1.05 g/mL, 1.1 g/mL, 1.15 g/mL, 1.2 g/mL, 1.25 g/mL, 1/3 g/mL, or ranges including and or spanning the aforementioned ranges. In some embodiments, the density of the composition is modified to approximately equal the density of an aqueous solution, a gel, a liquid, a cream, or a lotion. In some embodiments, the density is modified by adjust the ratio of different nanoparticle types (e.g., liposomes, solid lipid nanoparticles, etc.). In some embodiments, the density is modified by adjusting the ratio between a first lipid and second lipid. In some embodiments, the density is modified by adjusting the ratio between a first set of lipids and a second set of lipids. In some embodiments, concentrations of lipids with different chain lengths (e.g. 8 carbon, 10 carbon, 18 carbon lipids) are adjusted to modify the density. Such modifications described for modifying density may also influence API solubility (i.e., encapsulation), physical stability, chemical stability, particle composition

In some embodiments, the composition comprises 0.001 wt. % to 20 wt. % active agent, 5 wt. % to 20 wt. % HSPC, 1 wt. % to 15 wt. % MCT, 0-1% vitamin E, 0.2 wt. % to 2 wt. % cholesterol and/or plant sterol(s), and 66-67% trehalose. In some embodiments, the composition further comprises 0.01 wt. % to 5 wt. % of a gum. In some embodiments, the composition comprises 6-7% active agent, 27-28% lipid carrier, and 66-67% carbohydrate.

### A. Nanoparticles

In several embodiments, the particle comprises a lipid source, a surfactant, a co-emulsifier, or combinations of the foregoing. In several embodiments, the particle is a nanoscale particle (e.g., a nanoparticle). In several embodiments, the particle is a microscale particle (e.g., a microparticle).

In several embodiments, advantageously, the individual particles within the disclosed nanoparticle compositions may not settle or sediment appreciably. In several embodiments, an appreciable amount of the composition (e.g., as viewed by the naked eye) does not settle and/or separate from an aqueous liquid upon standing. In several embodiments, the composition does not appreciably settle or separate from an aqueous liquid upon standing for equal to or at least about 1 day, at least about 1 month, about 3 months, about 6 months, about 9 months, about 1 year, or ranges including and/or spanning the aforementioned values. In several embodiments, upon standing, the composition remains dispersed in an aqueous liquid for at least about 1 day, at least about 1 month, about 3 months, about 6 months, about 9 months, about 1 year, or ranges including and/or spanning the aforementioned values. In several embodiments, the homogeneity of the disclosed compositions changes by equal to or less than about: 0.5%, 1%, 5%, 7.5%, 10%, or 15% (or ranges including and/or spanning the aforementioned values) after a period of one week or one month. In this case, homogeneity is observed through images by SEM or cyro-SEM (e.g., the average size of the particles and/or the particle types). In several embodiments, the composition remains dispersed in an aqueous liquid and does not appreciably settle or separate from an aqueous liquid after at least about: 1 minute, 5 minutes, 30 minutes, or an hour in a centrifuge at a centripetal acceleration of at least about 100 m/s, at least about 1000 m/s, or at least about 10,000 m/s. In several embodiments, the composition remains dispersed in an aqueous liquid and does not appreciably settle or separate from an aqueous liquid after at least about: 1 minute, 5 minutes, 30 minutes, or an hour in a centrifuge at a centrifuge speed of 5000 RPM, 10,000 RPM, or 15,000 RPM.

In several embodiments, the average size of the nanoparticles of a composition as disclosed herein is substantially constant and/or does not change significantly over time (e.g., it is a stable nanoparticle). In several embodiments, after formulation and storage for a period of at least about 1 month (30 days), about 3 months (90 days), or about 6 months (180 days) (e.g., at ambient conditions, at 25° C. with 60% relative humidity, or under the other testing conditions disclosed elsewhere herein), the average size of nanoparticles comprising the composition changes less than or equal to about: 1%, 5%, 10%, 20%, or ranges including and/or spanning the aforementioned values.

In several embodiments, the average size of the nanoparticles of a composition as disclosed herein is substantially constant and/or does not change significantly over time (e.g., it is a stable nanoparticle) at higher temperatures, such as temperatures over 70° C. In several embodiments, after formulation and exposure to higher temperatures, the average size of nanoparticles comprising the composition changes less than or equal to about: 1%, 5%, 10%, 20%, or ranges including and/or spanning the aforementioned values. In several embodiments, after formulation and exposure to higher temperatures, the concentration of an active ingredient in the nanoparticles changes less than or equal to about: 1%, 5%, 10%, 20%, or ranges including and/or spanning the aforementioned values.

In several embodiments, the polydispersity index (PDI) of the nanoparticles of a composition as disclosed herein is less than or equal to about: 0.05, 0.10, 0.15, 0.20, 0.25, 0.30, 0.40, 0.50, 0.60, 0.70, 0.80, or ranges including and/or spanning the aforementioned values. In several embodiments, the size distribution of the nanoparticles is highly monodisperse with a polydispersity index of less than or equal to about: 0.05, 0.10, 0.15, 0.20, 0.25, or ranges including and/or spanning the aforementioned values.

In several embodiments, the zeta potential of the nanoparticles of a composition as disclosed herein is less than or equal to about: 1 mV, 3 mV, 4 mV, 5 mV, 6 mV, 7 mV, 8 mV, 10 mV, 20 mV, or ranges including and/or spanning the aforementioned values. In several embodiments, the zeta potential of the nanoparticles is greater than or equal to about: −3 mV, −1 mV, 0 mV, 1 mV, 3 mV, 4 mV, 5 mV, 6 mV, 7 mV, 8 mV, 4 mV, 10 mV, 20 mV, or ranges including and/or spanning the aforementioned values. In several embodiments, the zeta potential and/or diameter of the particles (e.g., measured using dynamic light scattering) is acquired using a zetasizer (e.g., a Malvern ZS90 or similar instrument).

In several embodiments, the nanoparticle composition has a pH of less than or equal to about: 2, 3, 4, 5, 6, 6.5, 7, 8, 9, or ranges including and/or spanning the aforementioned values. In several embodiments, the composition has a pH of greater than or equal to about: 2, 3, 4, 5, 6, 6.5, 7, 8, 9, or ranges including and/or spanning the aforementioned values. For example, in several embodiments, the composition has a pH ranging from 2 to 4, 4 to 6, 6 to 8, 2 to 9, 2 to 5, 3 to 7, 4 to 8, 5 to 9, etc.

In several embodiments, multilamellar nanoparticles comprise equal to or at least about 5%, 8%, 9%, 10%, 15%, 25%, 50%, 75%, 85%, 95%, or 100% (or ranges spanning and/or including the aforementioned values). of the particles present in the composition (e.g., the aqueous composition) For example, in some embodiments, between about 5% and about 10% of the particles present are multilamellar. In several embodiments, about 8.6% of the particles present are multilamellar.

In several embodiments, unilamellar nanoparticles comprise equal to or at least about 5%, 8%, 9%, 10%, 15%, 20%, 25%, 50%, 75%, 85%, 95%, or 100% (or ranges spanning and/or including the aforementioned values) of the particles present in the composition (e.g., the aqueous composition). For example, in some embodiments, between about 10% and about 15% of the particles present are unilamellar. In several embodiments, about 12.88% of the particles present are unilamellar.

In several embodiments, emulsion particles comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition). For example, in some embodiments, between about 60% to about 75% of the particles present are emulsion particles. In several embodiments, about 69.7% of the particles present are emulsion particles.

In several embodiments, micelle particles comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition).

In several embodiments, liposomes comprise equal to or at least about 5%, 8%, 9%, 10%, 15%, 25%, 50%, 60%, 65%, 70%, 75%, 85%, 95%, or 100% (or ranges spanning and/or including the aforementioned values) of the particles present in the composition (e.g., the aqueous composition).

In several embodiments, irregular particles (including particles with lamellar structures and/or bridges) comprise equal to or at least about 1%, 2%, 3%, 4%, 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition). For example, in some embodiments, between about 1% to about 5% of the particles present are irregular particles. In several embodiments, 2.73% are irregular particles.

In several embodiments, combined lamellar and emulsion particles comprise equal to or at least about 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition). For example, in some embodiments, between about 5% to about 6% of the particles present are combined lamellar and emulsion particles. In several embodiments, 6.06% of the particles are combined lamellar and emulsion particles.

In several embodiments, mixed-micelle particles comprise equal to or at least about 5%, 6%, 7%, 8%, 9%, 10%, 15%, 25%, 50%, 75%, 85%, 95%, or 100% (or ranges spanning and/or including the aforementioned values) of the particles present in the composition (e.g., the aqueous composition).

The nanoparticle compositions can comprise combinations of multilamellar nanoparticles, unilamellar nanoparticles, emulsion nanoparticles, micelle nanoparticles, irregular particles, and/or liposomes.

The percentages and/or concentrations of particles present in the composition may be purposefully modified. In some embodiments, the percentage and/or concentration of the particles present in the composition are tailored to the active compound and/or the liquid comprising the particles. Such tailoring may lead to more homogenization and/or dispersion in the liquid. The tailoring may stabilize dispersion in the liquid. Such tailoring may also tailor to specific densities of the compositions. The densities of the compositions can be matched or different from a liquid that the compositions are contacted by or contained within.

In some embodiments, the composition is biased towards one type of nanoparticle, such as solid nanoparticles or liposomes. The composition may be biased by increasing or decreasing the ratio in the composition of lipids that are solid at room temperature to lipids that are liquid at room temperature. Lipids that are liquid at room temperature may comprise MCT (a mixture of capric and caprylic triglycerides, which may have a ratio of c8:c10 carbon chains of 45:55), captex 1000 (a triglyceride of capric acid). Lipids that are solid at room temperature may comprise solubilizers and/or emollients. Lipids that are solid at room temperature may comprise phosphatidylcholine (such as HSPC) phosphatidylethanolamine, sphingomyelin, triglycerides of oleic acid, and/or triglycerol monooleate. In some embodiments, the concentration of an oil is adjusted, such as the concentration of a triglyceride, a fatty acid, a diglyceride, or a monoglyceride. In some embodiments, the concentration of a sterol, such as cholesterol or a plant sterol, is adjusted. In some embodiments, the composition is biased towards liposomes by increasing the concentration of lipids that are liquid at room temperature. Biasing the composition may alter characteristics of the composition including density, particle composition, solubility, pharmacokinetic properties, or other characteristics described herein.

Decreasing the concentration of a co-emulsifier that is a liquid at room temperature can bias the outcome of the particles more towards liposomes while increasing the concentration of a co-emulsifier that is a liquid at room temperature can bias the outcome of the particles more towards solid lipid nanoparticles. Also, increasing the concentration of a lipid that is solid at room temperature can bias the outcome of particles more towards liposomes. As non-limiting examples, decreasing concentrations of the co-emulsifier MCT and/or increasing the concentration of HSPC will bias the outcome of the particles more towards liposomes. As a non-limiting example, substituting MCT with a lipid that is a liquid at room temperature and/or increasing the concentration ratio of HSPC to MCT will bias the outcome of the particles more towards liposomes. As another non-limiting example, substituting HSPC with a lipid that is a solid at room temperature will bias the outcome of the particles more towards solid lipid nanoparticles.

In some embodiments, the composition comprises high purity triglycerides, such as oleic acid and/or conjugated linoleics. The composition may be formulated, such as by changing the composition or concentration of lipids, for specific delivery or specific metabolism. For example, the composition may comprise medium chain triglycerides to bias the composition towards phase 1 liver metabolism. In some embodiments, the composition is formulated for a specific absorption mechanism, such as lymphatic absorption or liver first pass.

Some embodiments pertain to a lipid-based particle composition comprising a nanoparticle comprising an active compound that is of sufficient purity that it exists in a solid and/or powdered state prior to formulation in the nanoparticle composition at a weight percent in the composition ranging from 0.001% to 20%; a phosphatidylcholine at a weight percent in the composition ranging from 2.5% to 20%; a sterol at a weight percent in the composition ranging from 0.2% to 5%; and a medium chain triglyceride at a weight percent in the composition ranging from 1% to 15%. In some embodiments, the composition comprises water at a weight percent in the composition ranging from 60% to about 95%. In some embodiments, the nanoparticles have an average size ranging from about 75 nm to about 175 nm. In some embodiments, upon storage for a period of one month, the average size of the nanoparticles changes by less than about 20%.

In some embodiments, the nanoparticle composition is in the form of liposomes and/or an oil-in-water nano-emulsion. In some embodiments, an appreciable amount of the nanoparticle composition does not settle and/or separate from the water upon standing for a period of at least about 12 hours, 24 hours, 3 days, 5 days, a week, 2 weeks, 3 weeks, 5 weeks, 2 months, 3 months, 6 months, 12 months, 18 months, or 24 months. In some embodiments, the composition is configured such that when concentrated to dryness to afford a powder formulation of nanoparticles, the nanoparticle powder can be reconstituted to provide the nanoparticle composition. In some embodiments, the composition has a Tmax for an active agent of less than 4.5 hours. In some embodiments, upon storage for a period of one month, two months, three months, 6 months, 12 months, 18 months, or 24 months the average size of the nanoparticles changes by less than about 20%. In some embodiments, the polydispersity of the nanoparticles in the composition is less than or equal to 0.15. In some embodiments, upon 90 days of storage at 25° C. and 60% relative humidity, the polydispersity of the nanoparticles changes by less than or equal to 10%. In some embodiments, upon 90 days of storage at 25° C. and 60% relative humidity, the polydispersity of the nanoparticles changes by less than or equal to 0.1. In some embodiments, the composition has a shelf life of greater than 18 months at 25° C. and 60% relative humidity. In some embodiments, upon 90 days of storage at 25° C. and 60% relative humidity, the D90 of the nanoparticles changes less than or equal to 10%. In some embodiments, the composition has a concentration max (Cmax) of 80 ng/ml or greater after an oral dose of 15 mg/kg.

In some embodiments, as disclosed elsewhere herein, the nanoparticle composition is in the form and/or comprises one or more of liposomes, an oil-in-water nano-emulsion (and/or microparticle emulsion), and/or solid lipid particles. In some embodiments, when suspended in water, an appreciable amount of the particles in the composition do not settle and/or do not separate (e.g., upon visual inspection) from the water upon standing for a period of at least about 12 hours. In some embodiments, when suspended in water, the particles remain substantially homogenously distributed in the water upon standing for a period of at least about 12 hours, 24 hours, 3 days, 5 days, a weck, 2 weeks, 3 weeks, 5 weeks, 2 months, 3 months, 6 months, 12 months, 18 months, or 24 months. In some embodiments, the nanoparticles have an average size ranging from about 10 nm to about 500 nm. In some embodiments, the composition comprises nanoparticles having an average size of less than or equal to about: 10 nm, 50 nm, 100 nm, 250 nm, 500 nm, 1000 nm, or ranges including and/or spanning the aforementioned values. In some embodiments, the composition comprises microparticles having an average size of less than or equal to about: 1000 nm, 1.5 μm, 2 μm, 3 μm, 5 μm, 10 μm or ranges including and/or spanning the aforementioned values. In some embodiments, the dried powder composition comprises microparticles that form nanoparticles (as disclosed herein) when reconstituted. In some embodiments, these dried powder compositions comprise particles having an average size of less than or equal to about: 250 nm, 500 nm, 1000 nm, 1.5 μm, 2 μm, 3 μm, 5 μm, 10 μm, 50 μm, or ranges including and/or spanning the aforementioned values. In some embodiments, upon storage for a period of one month, the average size of the nanoparticles (or microparticles) increases by less than about 10%.

In some embodiments, the nanoparticle composition is configured such that when concentrated to dryness to afford dry particles (e.g., from any one of the oil-in-water emulsion (e.g., a nanoemulsion or microemulsion), liposome solution, and/or solid lipid particle) as a powder, the dry nanoparticles can be reconstituted to provide a reconstituted particle based solution (e.g., the nanoparticle composition). In some embodiments, when reconstituted, the average size of the nanoparticles increases or decreases by less than about 15%, 20%, 25%, 30%, 35%, 40%, 45%, 50%, 55%, 60%, 65%, 70%, 75%, 80%, 85%, 90%, 95%, and/or by less than about 100%. In some embodiments, to form powders, excipients (and/or additives as disclosed elsewhere herein) may be added to the liposomes, oil-in-water nano-emulsions (and/or microparticle emulsions), and/or a solid lipid particle. In some embodiments, the excipient comprises a carbohydrate, such as trehalose.

### B. Active Compounds/Active Agents

Certain embodiments herein concern active compounds, also referred to as active agents. The active compounds may be encapsulated in one or more of the nanoparticles described herein. The active compounds may be pharmaceuticals, nutraceuticals, cosmetics, pigments, flavorings, and the like.

In some embodiments, the active compound comprises a therapeutic. The therapeutic may be an analgesic, an anesthetic, an antibacterial agent, an anticonvulsant, an antidementia agent, an antidepressant, an antidote, a deterrent, a toxicologic agent, an antiemetic, an antifungal, an antigout agent, an anti-inflammatory agent, an antimigraine agent, an antimyasthenic agent, an antincoplastic agent, an antiparasitic agent, an antiparkinson agent, an antipsychotic, an antipasticity agent, an antiviral, an anxiolytic, a bipolar agent, a blood glucose regulator, a blood product, a blood modifier, a blood volume expander, a cardiovascular agent, a central nervous system agent, a dental agent, an oral agent, a dermatological agent, an enzyme replacement agent, an enzyme modifying agent, a gastrointestinal agent, a genitourinary agent, a hormonal agent, a hormone stimulant, a hormone replacement, a hormone modifying agent, a hormone suppressant, an immunological agent, an inflammatory bowel disease agent, a metabolic bone disease agent, an ophthalmic agent, an otic agent, a respiratory tract agent, a sedative, a hypnotic, a skeletal muscle relaxant, a therapeutic nutrient, a therapeutic mineral, and/or a therapeutic electrolyte. The hormonal agent, a hormone stimulant, a hormone replacement, a hormone modifying agent, and/or hormone suppressant may act on the adrenal system, the pituitary system, the prostaglandin system, sex hormone, the thyroid, and/or the parathyroid.

The active compound may comprise a small molecule. The active compound may comprise a biologic. The active compound may comprise a biomolecule. The active compound may comprise a macromolecule. In some embodiments, the active compound comprises a nucleic acid, a protein, a lipid, a carbohydrate, or a combination thereof. In some embodiments, the active compound comprises a cell or a derivative of a cell. In some embodiments, the active compound comprises antisense RNA. In some embodiments, the active compound comprises an siRNA, a miRNA, a IncRNA, or a combination thereof. In some embodiments, the active compound comprises a nucleic acid vector.

In some embodiments, the active compound is one or more of a cannabinoid, cannabidiol, cannabigerol, cannabinol, cannabichromene, tetrahydrocannabivarin, tetrahydrocannabinol, full extracts of hemp, specific ratios of isolated cannabinoids, cannabigerolic acid, cannabidolic acid, mitragynine, payantheine, mitraphylline, speciociliantine, speciogynine, cholecalciferol, ergocalciferol, D,L-alpha-tocopherol, menaquinone, ascorbyl palmitate, retinyl palmitate, beta-sitosterol, plant sterol rich extracts, cholesterol, ubiquinone, phosphatidylcholine, phosphatidylserine, eicosapentaenoic/docosahexaenoic acid mixtures, oleic acid, conjugated linoleic acid, capric triglycerides, caprylic triglycerides, capric and caprylic triglyceride mixtures, peppermint, orange, lemon oils, lutein, kavain, methysticin, yangonin, dihydromethysticin, coenzyme Q10, a vitamin E, a beta carotene, squalene, a vitamin K, a docosahexaenoic acid, a curcuminoid, a phytoceramide, vitamin D3, an ashwagandha extract, or any combination thereof.

In some embodiments, the agent is selected from the group consisting of Noopept (N-phenylacetyl-L-prolyglygice ethyl ester), melatonin, glutathione, gamma-glutamylcysteine (GGC), gamma-aminobutyric acid (GABA), valerian root, magnesium, theanine, 5-HTP, tyrosine, taurine, zinc, alpha fenchone, alpha terpinene, alpha terpineol, beta caryophyllene, alpha pinene, beta pinene, bisabolene, bisabolol, borneol, eucalyptol, gamma terpinene, guaiacol, humulene, linalool, myrcene, para cymene, phytol, terpinolene, limonene, others, and/or combinations thereof.

In some embodiments, the active compound may be prepared using the thoroughness and diligence of pharmaceutical drug development to consumer products. In several embodiments, when a hydrophilic composition is used, it is mixed with the aqueous soluble ingredients before mixing with the lipid ingredients.

In some embodiments, the active compound comprises at least one cosmetic ingredient. The CTFA International Cosmetic Ingredient Dictionary and Handbook (2004 and 2008) describes a wide variety of non-limiting cosmetic ingredients that can be used in the context of the present disclosure, including as active compounds. In some embodiments, the active compound comprises a fragrance, flavor, dye, etc. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatoires), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben, mushroom extract, etc.), pH adjusters (e.g., sodium hydroxide, ascorbic acid, and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloc extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate).

In some embodiments, the active compound includes at least one UV absorption and/or reflecting agent. UV absorption and/or reflecting agents that can be used in combination with the compositions of the present disclosure include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, cthyl PABA, cthyl dihydroxypropyl PABA, benzophenones (oxybenzone, sulisobenzone, benzophenone, and benzophenone-1 through 12), cinnamates (octyl methoxycinnamate (octinoxate), isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloyl trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazinc, 4-methylbenzylidenc camphor, and isopentyl 4-methoxycinnamate. Non-limiting examples of physical sunblocks include, kaolin, talc, petrolatum and metal oxides (e.g., titanium dioxide and zinc oxide).

In some embodiments, the active compound comprises at least one moisturizing agent. Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honcy, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol. Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, crythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan perolcate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang ylang (Cananga odorata) oil.

In some embodiments, the active compound comprises at least one antioxidant. Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCI, diamylhydroquinone, di-t-butylhydroquinone, dicetyl thiodipropionate, diolcyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In some embodiments, the active compound comprises at least one essential oil. Essential oils include oils derived from herbs, flowers, trees, and other plants. Such oils are typically present as tiny droplets between the plant's cells, and can be extracted by several methods known to those of skill in the art (e.g., steam distilled, enfleurage, maceration, solvent extraction, or mechanical pressing). When these types of oils are exposed to air they tend to evaporate. As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160 to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

In some embodiments, the active agent comprises an algae extract. The algae extract may comprise ashwagandha and/or astaxanthin.

In some embodiments, the active compound is encapsulated by a nanoparticle at a concentration of 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, or ranges including and/or spanning the aforementioned values, per kg of the nanoparticle.

### C. Pharmacokinetic Properties

In several embodiments, as disclosed elsewhere herein, the nanoparticle composition aids in absorption, bioavailability, or other pharmacokinetic properties of the active compound when administered to an individual, including by orally ingestion. In several embodiments, the compositions disclosed herein allow the active compound to be delivered to and/or absorbed through the gut. As disclosed elsewhere herein, some embodiments pertain to the use of the nanoparticle based nanodelivery system to protect the active compound from degradation and/or precipitation in a solution comprising the active compound (e.g., in an aqueous composition for administration to a subject). In several embodiments, use of the delivery systems, including the nanoparticles, disclosed herein result in improved bioavailability and/or absorption rate. For instance, in some embodiments, the Cmax of an active compound is increased using a disclosed embodiment, the Tmax of an active compound is decreased using an embodiment as disclosed herein, and/or the AUC of an active compound is increased using a disclosed embodiment.

In several embodiments, the pharmacokinetic outcomes disclosed elsewhere herein (Cmax, Tmax, AUC, _{t1/2}, etc.) can be achieved using aqueous nanoparticle compositions or powdered nanoparticle compositions (e.g., where the powder is supplied by itself, in a gel capsule, as an additive to food, etc.).

In several embodiments, the Cmax of the active agent is increased using the disclosed embodiments relative to other delivery vehicles (e.g., after administration to a subject). In several embodiments, the Cmax is increased relative to the active agent (e.g., pharmaceuticals, nutraceuticals, and the like) alone or comparator embodiments (e.g., oil-based products) by equal to or at least about: 15%, 20%, 50%, 100%, 150%, 200%, or ranges including and/or spanning the aforementioned values. In several embodiments, the active agent Cmax is increased (relative to a comparator oil-based product) by equal to or at least about: 5%, 10%, 20%, 30%, 50%, 100%, or ranges including and/or spanning the aforementioned values. In several embodiments, the active agent Cmax is increased (relative to a comparator oil-based product) by equal to or at least about: 10 ng/ml, 20 ng/ml, 30 ng/ml, 40 ng/ml, 50 ng/mL, 60 ng/ml, 70 ng/ml, 80 ng/mL, 90 ng/mL, or ranges including and/or spanning the aforementioned values.

In several embodiments, after a dose of 15 mg of active agent (e.g., pharmaceuticals, nutraceuticals, and the like) provided in an embodiment as disclosed herein to a subject (e.g., a mini-pig, human, etc.), the Cmax of the active agent is equal to or at least about: 0.5 μg/L, 1 μg/L, 2 μg/L, 3 μg/L, 4 μg/L, 5 μg/L, 6 μg/L, or ranges including and/or spanning the aforementioned values. In several embodiments, after a dose of 15 mg/kg of active agent (e.g., pharmaceuticals, nutraceuticals, and the like) provided in an embodiment as disclosed herein to a subject, the Cmax is equal to or at least about: 40 ng/ml, 50 ng/ml, 60 ng/mL, 70 ng/mL, 80 ng/mL, 90 ng/mL, 100 ng/ml, 150 ng/mL, 200 ng/mL, or ranges including and/or spanning the aforementioned values.

In several embodiments, the Cmax for a disclosed embodiment is increased relative to an equal dose of an active agent (e.g., pharmaceuticals, nutraceuticals, and the like) in an oil-based comparator vehicle. In several embodiments, the Cmax for a disclosed embodiment is increased relative to an oil-based comparator vehicle by equal to or at least about: 15%, 20%, 50%, 100%, 150%, 200%, or ranges including and/or spanning the aforementioned values. In several embodiments, these pharmacokinetic results can be achieved using aqueous compositions or powdered compositions (where the powder is supplied by itself, in a gel capsule, as an additive to food, etc.). In some instances, the Cmax using a disclosed embodiment is 1.25 times higher than when using a comparator delivery system (e.g., the Cmax of the comparator×1.25). In some instances, the Cmax using a disclosed embodiment is equal to or at least about 1.25 times higher, 1.5 times higher, 2 times higher, 3 times higher (or ranges including or spanning the aforementioned values) than when using a comparator delivery system.

In several embodiments, the Tmax for an active agent using a disclosed embodiment is shortened relative to other vehicles. In several embodiments, after a dose of active agent (e.g., pharmaceuticals, nutraceuticals, and the like) provided in an embodiment as disclosed herein to a subject as disclosed herein, the Tmax is equal to or at less than about: 30 minutes, 1 hours, 2 hours, 3 hours, 4 hours, 4.5 hours, 5 hours, 5.5 hours, 6 hours, 6.5 hours, 7 hours, 8 hours, or ranges including and/or spanning the aforementioned values. In several embodiments, after a dose of 15 mg/kg of active agent provided in an embodiment as disclosed herein to a subject, the Tmax is equal to or at less than about: 30 minutes, 1 hours, 2 hours, 3 hours, 4 hours, 5 hours, 5.5 hours, 6 hours, 6.5 hours, 7 hours, 8 hours, or ranges including and/or spanning the aforementioned values. In several embodiments, after a dose of active agent provided in an embodiment as disclosed herein to a subject, the Tmax is between about 4 hours and about 6.5 hours or between about 3 hours and about 7 hours. In several embodiments, after a dose of 15 mg of active agent provided in an embodiment as disclosed herein to a human patient, the Tmax is equal to or less than about: 3 hours, 4 hours, 5 hours, 6 hours, 7 hours, 8 hours, or ranges including and/or spanning the aforementioned values.

In several embodiments, after of a dose of active agent (e.g., pharmaceuticals, nutraceuticals, and the like) (e.g., a 15 mg/kg dose) provided in an embodiment as disclosed herein to a subject (e.g., a mini-pig, human, etc.), the AUC is equal to or at least about: 50 ng/mL*hr, 100 ng/mL*hr, 200 ng/mL*hr, 300 ng/mL*hr, 400 ng/mL*hr, 450 ng/mL*hr, 500 ng/mL*hr, 550 ng/mL*hr, 600 ng/mL*hr, 650 ng/mL*hr, 700 ng/mL*hr, 800 ng/mL*hr, 1000 ng/mL*hr, or ranges including and/or spanning the aforementioned values.

In several embodiments, the half-life for an active agent (e.g., pharmaceuticals, nutraceuticals, and the like) (t_{1/2}) in vivo using a disclosed embodiment can be shorter relative to other vehicles. In several embodiments, after a dose of active agent (e.g., pharmaceuticals, nutraceuticals, and the like) provided in an embodiment as disclosed herein to a subject as disclosed herein, the tin of active agent is equal to or at less than about: 4 hours, 5 hours, 5.5 hours, 6 hours, 6.5 hours, or ranges including and/or spanning the aforementioned values. In several embodiments, after a dose of active agent provided in an embodiment as disclosed herein to a subject, the tin of active agent is between about 4 hours and about 6.5 hours or between about 3 hours and about 7 hours. In several embodiments, the t_{1/2} for a disclosed embodiment is decreased relative to an active agent alone or an oil-based comparator vehicle by equal to or at least about: 15%, 20%, 50%, 100%, 150%, 200%, or ranges including and/or spanning the aforementioned values. In several embodiments, the t_{1/2} of active agent for a disclosed embodiment is decreased relative to the active alone or an oil-based comparator vehicle by equal to or at least about: 15 minutes, 30 minutes, 45 minutes, 1 hour, 2 hours, or ranges including and/or spanning the aforementioned values. In some instances, the tin is a fraction of that achieved using a comparator delivery system. In some instances, the time to tin using a disclosed embodiment is 0.5 times, 0.7 times, 0.8 times, 0.9 times, or 0.95 times the tin of a comparator delivery system (or ranges including or spanning the aforementioned values).

In several embodiments, as disclosed elsewhere herein, the nanoparticle composition is stable. In several embodiments, for example, after formulation (e.g., in water at concentrations disclosed elsewhere herein) and storage for a period of at least about 1 month, 3 months, 6 months, 12 months, 18 months, 24 months, or ranges including or spanning the aforementioned values, the polydispersity of the nanoparticles changes less than or equal to about: 1%, 5%, 10%, 20%, or ranges including and/or spanning the aforementioned values. In several embodiments, after formulation (e.g., in water at concentrations disclosed elsewhere herein) and storage for a period of at least about 1 month, 3 months, 6 months, 12 months, 18months, 24 months, or ranges including or spanning the aforementioned values, the soluble fraction of active agent (e.g., pharmaceuticals, nutraceuticals, cosmetics, pigments, flavorings, and the like) in the formulation changes less than or equal to about: 1%, 5%, 10%, 20%, 30%, or ranges including and/or spanning the aforementioned values. In several embodiments, after formulation and storage for a period of at least about 1 month, 3 months, 6 months, 12 months, 18 months, 24 months, or ranges including or spanning the aforementioned values, (e.g., at ambient conditions, at 25° C. with 60% relative humidity, or under the other testing conditions disclosed elsewhere herein), the PDI of nanoparticles comprising the composition changes by less than or equal to about: 1%, 5%, 10%, 20%, or ranges including and/or spanning the aforementioned values. In several embodiments, after formulation and storage for a period of at least about 1 month, 3 months, 6 months, 12 months, 18 months, 24 months, or ranges including or spanning the aforementioned values, (e.g., at ambient conditions, at 25° C. with 60% relative humidity, or under the other testing conditions disclosed elsewhere herein), the PDI of nanoparticles comprising the composition changes by less than or equal to about: 0.05,0.1, 0.2, 0.3, 0.4, or ranges including and/or spanning the aforementioned values.

In several embodiments, when exposed to simulated gastric fluid (e.g., at a concentration of 20 mg/mL), the particle size of the nanoparticles of a composition as disclosed herein does not change and/or changes less than 5% during a period of greater than or equal to about: 1 hour, 2 hours, 3 hours, 4 hours, 5 hours, 6 hours, 10 hours, or ranges including and/or spanning the aforementioned values. In several embodiments, when exposed to simulated intestinal fluid (e.g., at a concentration of 20 mg/mL), the particle size of the nanoparticles disclosed herein does not change and/or changes less than 5% during a period of greater than or equal to about: 1 hour, 2 hours, 3 hours, 4 hours, 5 hours, 6 hours, 10 hours, or ranges including and/or spanning the aforementioned values. In several embodiments, after formulation (e.g., at a concentration of 20 mg/mL) and storage in simulated gastric fluid for a period of at least about 1 hour or about 2 hours (e.g., at 37° C., or under the other testing conditions disclosed elsewhere herein), the average particle size of nanoparticles comprising the composition changes by less than or equal to about: 1%, 5%, 10%, 20%, 50%, or ranges including and/or spanning the aforementioned values. In several embodiments, after formulation (e.g., at a concentration of 20 mg/mL) and storage in simulated gastric fluid for a period of at least about 1 hour, about 2 hours, about 3 hours, or about 4 hours (e.g., at 37° C. or under the other testing conditions disclosed elsewhere herein), the PDI of nanoparticles comprising the composition changes by less than or equal to about: 1%, 5%, 10%, 20%, or ranges including and/or spanning the aforementioned values. In several embodiments, after formulation (e.g., at a concentration of 20 mg/mL) and storage in simulated gastric fluid for a period of at least about 1 hour or about 2 hours (e.g., at 37° C. or under the other testing conditions disclosed elsewhere herein), the PDI of nanoparticles comprising the composition changes by less than or equal to about: 0.01, 0.05, 0.1, 0.2, 0.3, or ranges including and/or spanning the aforementioned values. In several embodiments, after formulation (e.g., at a concentration of 20 mg/mL) and storage in simulated intestinal fluid for a period of at least about 1 hour or about 2 hours (e.g., at 37° C., or under the other testing conditions disclosed elsewhere herein or under the other testing conditions disclosed elsewhere herein), the average particle size of nanoparticles comprising the composition changes by less than or equal to about: 1%, 5%, 10%, 20%, 50%, or ranges including and/or spanning the aforementioned values. In several embodiments, after formulation (e.g., at a concentration of 20 mg/mL) and storage in simulated intestinal fluid for a period of at least about 1 hour, about 2 hours, about 3 hours, or about 4 hours (e.g., at 37° C. or under the other testing conditions disclosed elsewhere herein), the PDI of nanoparticles comprising the composition changes by less than or equal to about: 1%, 5%, 10%, 20%, 100%, 150%, or ranges including and/or spanning the aforementioned values. In several embodiments, after formulation (e.g., at a concentration of 20 mg/mL) and storage in simulated intestinal fluid for a period of at least about 1 hour, about 2 hours (e.g., at 37° C. or under the other testing conditions disclosed elsewhere herein), the PDI of nanoparticles comprising the composition changes by less than or equal to about: 0.01, 0.05, 0.1, 0.2, 0.3, or ranges including and/or spanning the aforementioned values.

In several embodiments, the composition particle size remains consistent (a size change of less than or equal to about: 0%, 0.5%, 1%, 2%, 3%, 5%, or ranges including and/or spanning the aforementioned values) for a period of at least about 30 days when stored at room temperature, refrigeration, and up to 40° C. In several embodiments, the active agent (e.g., pharmaceuticals, nutraceuticals, cosmetics, pigments, flavorings, and the like) concentration in the composition remains consistent (a loss of less than or equal to about: 0.5%, 1%, 2%, 3%, 5%, or ranges including and/or spanning the aforementioned values) for a period of at least about 30 days, 60 days, 90 days, or 120 days when stored at room temperature, refrigeration, and up to 40° C. In several embodiments, when stored at room temperature, refrigeration, and up to 40° C., the composition is stable (e.g., the particle size or active agent concentration in the nanoparticles remains consistent and/or has a change of less than or equal to about: 0.5%, 1%, 2%, 5%, or ranges including and/or spanning the aforementioned values) for a period of at least about: 2 weeks, 30 days, 2 months, 3 months, 6 months, 9 months, 1 year, or ranges including and/or spanning the aforementioned measures of time.

In several embodiments, when exposed to higher temperatures, e.g., above 70° C., the average size of nanoparticles comprising the composition changes less than or equal to about: 1%, 5%, 10%, 20%, or ranges including and/or spanning the aforementioned values. In several embodiments, after exposure to higher temperatures, the concentration of an active ingredient in the nanoparticles changes less than or equal to about: 1%, 5%, 10%, 20%, or ranges including and/or spanning the aforementioned values.

In several embodiments, the method of using the nanoparticle composition and/or of treating a subject with the nanoparticle composition includes administering to a subject in need of treatment (e.g., orally, topically, etc.) an effective amount of the composition. In several embodiments, the composition (e.g., delivery system) improves the stability of the active agent (e.g., pharmaceuticals, nutraceuticals, cosmetics, pigments, flavorings, and the like) after ingestion where the composition is exposed to the stomach and/or intestines in an aqueous environment with harsh pH conditions. In several embodiments, the bioavailability of the active agent (e.g., pharmaceuticals, nutraceuticals, cosmetics, pigments, flavorings, and the like) relative to the initial administered dose is greater than or equal to about: 10%, 20%, 50%, 75%, or ranges including and/or spanning the aforementioned values. In several embodiments, using the disclosed compositions, the oral bioavailability of the active agent (e.g., pharmaceuticals, nutraceuticals, cosmetics, pigments, flavorings, and the like) delivered (as measured using AUC) is higher using an embodiment disclosed herein relative to oral delivery of the active alone. In several embodiments, the oral bioavailability is improved over the active alone by greater than or equal to about: 10%, 50%, 75%, 100%, 200%, or ranges including and/or spanning the aforementioned values.

In other embodiments, solutions of particles are composed of non-lipid ingredients, such as polymers and/or cyclodextrin.

### III. Manufacturing Compositions

As disclosed elsewhere herein, some embodiments pertain to methods of preparing nanoparticle compositions. In several embodiments, the composition is prepared by adding one or more of an active compound, a lipid source, a surfactant, a co-emulsifier, a preservative, a flavoring agent, or combinations of any of the foregoing to water. In several embodiments, the composition is prepared using high sheer inline mixing (including for example via Silverson). In several embodiments, the composition is prepared using an overhead mixer (such as, for example, a IKA and/or Silverson). In several embodiments, the composition is prepared using high pressure homogenization. In several embodiments, the composition is prepared using microfluidization. In several embodiments, the composition is prepared using sonication. In several embodiments, the composition is prepared using mechanical stirring. In several embodiments, the composition is prepared using coacervation. In several embodiments, the composition is prepared using solvent precipitation. In several embodiments, the composition is prepared using hot melt extrusion (HME) tablet manufacturing. In several embodiments, the composition is prepared using one or more of the techniques or steps described above or elsewhere herein together. In several embodiments, the composition is prepared with methods excluding any one or more of these steps or techniques.

In several embodiments, the nanoparticle compositions herein are lyophilized (e.g., to provide a powder). In several embodiments, where lyophilization is used to prepare a mixed micelle-based powder, one or more lyoprotectant agents may be added. In several embodiments, an individual lyoprotectant agent may be present at a dry wt. % equal to or less than the dry weight of the lipophilic ingredients. In several embodiments, the lyoprotectant agent(s) (collectively or individually) may be present at a dry wt. % equal to or less than about: 5%, 10%, 15%, 20%, 30%, 40%, 50%, 60%, or ranges including and/or spanning the aforementioned values. In several embodiments, the lyoprotectant agent(s) (collectively or individually) may be present at a wet wt. % of equal to or less than about: 2.5%, 5%, 7.5%, 10%, 12.5%, 15%, 20%, 30%, or ranges including and/or spanning the aforementioned values. In several embodiments, the lyoprotectant is selected from the group consisting of lactose, dextrose, trehalose, arginine, glycine, histidine, and/or combinations thereof. In several embodiments, the nanoparticle compositions herein are spray dried (e.g., to provide a powder). In several embodiments, the nanoparticle compositions are spray dried and not lyophilized. In some embodiments, the nanoparticle composition is spray dried, fluid bed dried, desiccated, and/or lyophilized.

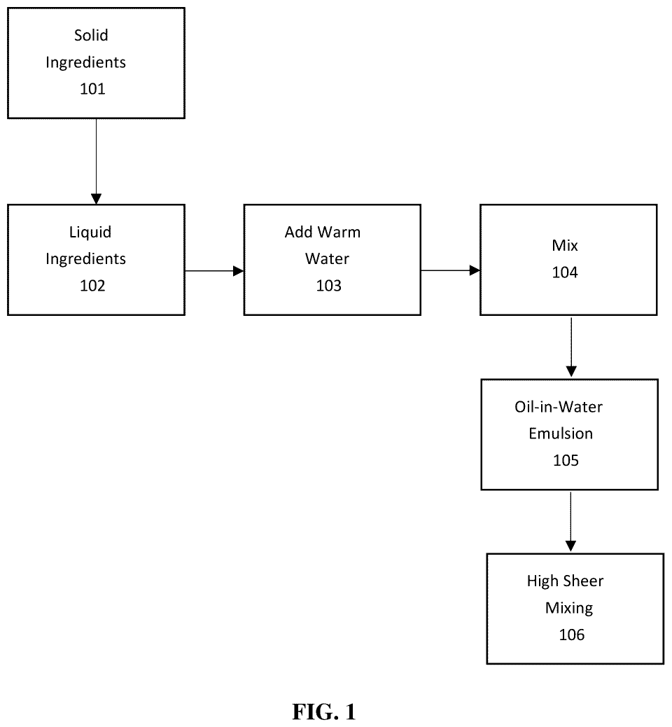

In several embodiments, the composition is prepared by forming a lipid-in-oil emulsion. In several embodiments, an oil-in-water emulsion can be prepared without the use of organic solvents as shown in FIG. 1 (e.g., in an organic solvent-free method). In several embodiments, solid ingredients 101 are added and dissolved into liquid ingredients 102. In several embodiments, the lipid (e.g., phosphatidylcholine, including phosphatidylcholine of any purity disclosed herein) can be added with mixing. In several embodiments, when a well dispersed lipid phase is formed after mixing, the addition of water 103 (e.g., having a temperature of equal to or at least about: 10° C., 20° C., 30° C., 40° C., 50° C., 60° C., 80° C., or ranges including and/or spanning the aforementioned values) and additional mixing 104 achieves an oil-in-water emulsion 105. In several embodiments, the oil-in-water emulsion is then subject to high-shear mixing to form nanoparticle compositions. In several embodiments, high-shear mixing 106 is performed using a high shear dispersion unit or an in-line mixer can be used to prepare the emulsions. In several embodiments, the particles can be made by solvent evaporation and/or solvent precipitation. In several embodiments, high sheer mixing is not required.

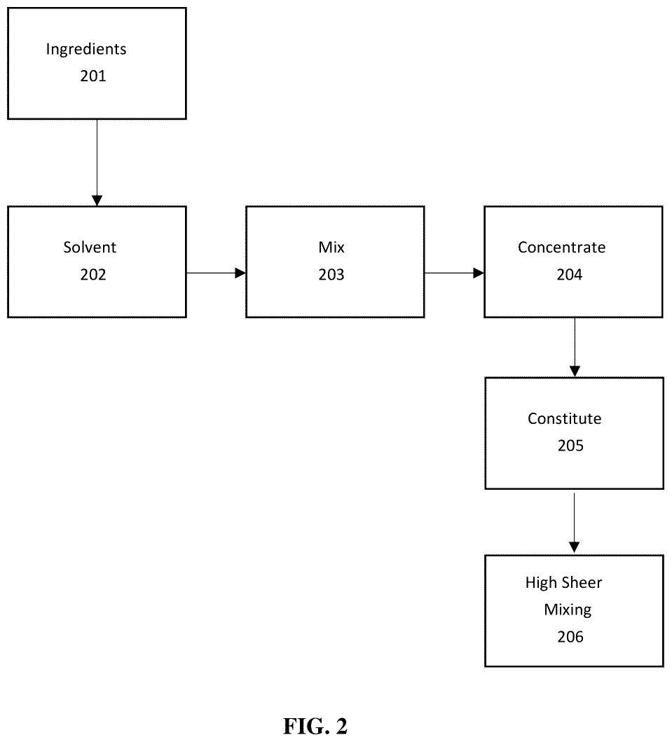

In several embodiments, as shown in FIG. 2, the lipid-in-oil emulsion is formed by dissolving ingredients 201, such as, one or more of a lipid (e.g., phosphatidylcholine, including phosphatidylcholine or any purity disclosed herein), a surfactant, one or more active compounds, and/or a preservative in a solvent 202. In several embodiments, the solvent can include one or more organic solvents, including but not limited to, ethanol, chloroform, and/or ethyl acetate. In several embodiments, the solvents are class II solvents, class III solvents (e.g., at least class II and/or class III by the ICH Q3C standard), or mixtures thereof. In several embodiments, the solution of ingredients and solvent is dried 203. In several embodiments, after drying, the ingredients are provided as lipids and or liposomes as a thin film. In several embodiments, the solvent is removed from the composition by subjecting the solution to heat under vacuum to promote evaporation. In several embodiments, the film may further be dried under nitrogen gas. In several embodiments, the lipid film is hydrated 205 with warm aqueous solution to form an oil-in-water emulsion. In several embodiments, high-shear mixing is performed 206 using a high shear dispersion unit or an in-line mixer can be used to prepare the emulsions.

In some embodiments, the dried composition, comprising the nanoparticle, is reconstituted. In some embodiments, the nanoparticle composition, such as the percentage and/or concentration of the types of nanoparticles, may change when dried. In some embodiments, the nanoparticle composition, such as the percentage and/or concentration of the types of nanoparticles, may change when reconstituted. In some embodiments, the nanoparticle composition, such as the percentage and/or concentration of the types of nanoparticles, may not change when dried. In some embodiments, the nanoparticle composition, such as the percentage and/or concentration of the types of nanoparticles, may not change when reconstituted.

In several embodiments, as disclosed elsewhere herein, the lipid-in-water emulsion is subject to high pressure homogenization using a microfluidizer. In several embodiments, high sheer mixing can be used to reduce the particle size. In several embodiments, the oil-in-water emulsion is processed to a nanoparticle (e.g., about 20 to about 500 nm, etc.) using the microfluidizer or other high sheer processes. In several embodiments, the oil-in-water emulsion is processed to a nanoparticle having a size from about 80 nm to 180 nm in diameter or about 100 nm to about 150 nm in diameter. In several embodiments, high sheer mixing is not used.

In several embodiments, the lipid-in-water emulsion is passed through the microfluidizer a plurality of times (e.g., equal to or at least 1 time, 2 times, 3 times, 4 times, 5 times, 10 times, or ranges including and/or spanning the aforementioned values). In several embodiments, the emulsion is passed through the microfluidizer at a pressure of equal to or less than about: 5,000 PSI, 15,000 PSI, 20,000 PSI, 25,000 PSI, 30,000 PSI, or ranges including and/or spanning the aforementioned values. In several embodiments, the emulsion is passed through the microfluidizer at a temperature of equal to or at least about: 30° C., 40° C., 50° C., 65° C., 80° C., or ranges including and/or spanning the aforementioned values. In several embodiments, the emulsion is passed through the microfluidizer at least about room temperature (e.g., about 20° C. or about 25° C.) and/or without any heating and/or temperature control. In several embodiments, the emulsion is passed through the microfluidizer at a temperature of equal to or less than about 80° C. In several embodiments, the microfluidizer includes an interaction chamber consisting of 75 μm to 200 μm pore sizes and the emulsion is passed through this chamber. In several embodiments, the pore size of the microfluidizer are less than or equal to about: 75 μm, 100 μm, 150 μm, 200 μm, 250 μm, 300 μm, or ranges including and/or spanning the aforementioned values. In several embodiments, the nanoparticle composition is prepared by high shear mixing, sonication, or extrusion.

In several embodiments, after preparation, the nanoparticle composition is characterized by an ability to pass through a 0.2 um filter while preserving the nanoparticle structure (e.g., a change in average nanoparticle size of no greater than 10 nm, 20 nm, or 30nm). In several embodiments, after passage through a 0.2 μm there is a change in average diameter of the particles of equal to or at less than about: 1%, 5%, 10%, 20%, or ranges including and/or spanning the aforementioned values. In several embodiments, after passage through a 0.2 μm there is a change in PDI of the particles of equal to or at less than about: 1%, 5%, 10%, 20%, or ranges including and/or spanning the aforementioned values.

In several embodiments, as disclosed elsewhere herein, the active nanoparticle composition imparts solubility to hydrophobic active agents in a delivery system that is easily dispersible in aqueous solutions.

Some embodiments, as disclosed elsewhere herein, pertain to a method of manufacturing a lipid-based particle composition. In some embodiments, one or more active compounds is mixed with one or more lipophilic components of the composition to provide a solution. In some embodiments, one or more lipid components (including those that are not phospholipids) are added. In some embodiments, one or more sterols are added. In some embodiments, one or more phospholipids are added. In some embodiments, one or more flavoring and/or preservatives are added. In some embodiments, water is added. In some embodiments, the lipophilic ingredients are combined and the hydrophilic ingredients are combined separately. In some embodiments the lipophilic ingredients are then added to the hydrophilic ingredients. In some embodiments, the solution is passed through a microfluidizer and/or a high sheer homogenizer. In some embodiments, the process affords a particle composition.

In several embodiments, advantageously, the nanoparticle delivery systems disclosed herein are reproducibly manufacturable.

In several embodiments, the active compound may be preprocessed. In several embodiments, pre-processing allows greater encapsulation efficiency and stability by precipitating “other” plant material away from extracts. In several embodiments, pre-processing may provide enhanced consumer experience due to less impurity/non-actives in the formulation. In several embodiments, “salt” byproducts are removed directly/indirectly by various extraction techniques. In several embodiments, pre-processing techniques include ethanol and co-solvent precipitation, filtration, activated charcoal soaking+filtration; chromatography, etc. By way of removing solvent such as ethanol, rotary evaporation may be used. Other forms of solvent removal are possible. Other forms of removing active and non-active plant material are disclosed elsewhere herein (including solvent extraction, acid/base titration, CO₂ extraction (both supercritical and non), cryogenic ethanol extraction, etc. In several embodiments, pre-processing (prior to use in the composition) allows a formulator to use any kind of plant extract or biomass regardless of prior extraction techniques.

Other appropriate methods of salt separation and/or purification during preprocessing may include one or more of size exclusion, ion exchanger in presence of neutral organic compounds may also be a suitability method of purifications, evaporation and distillation membrane extraction, liquid-liquid extraction, solid phase extraction, immobilized liquid extraction, sorptive extraction, charged resin, and/or gel filtration. In several embodiments, pharmaceutical acceptable applications may also be used such as high purity filter media such as diatomite filter aids. Example may include Celpure®, AW Celite®, Harborlite®, etc. In several embodiments, dialysis methods may also be used. In several embodiments, activated charcoal is used to treat a solution comprising the extract. In several embodiments, acidic conditions may be used for preprocessing. In several embodiments, polar, aprotic solvent like DMSO may also be used to solubilize hydrophobic substances like alkaloids during preprocessing. In several embodiments, DMSO and may be acidified or basified to maximize solubility and stability of alkaloids and other compounds.

One or more benefits of the preprocessing step may include better formulations, better encapsulation, purer compositions, industrial isolation for raw material, certified reference material/standards, manufacturer of finished intermediate and/or raw materials, or other benefits.

In several embodiments, the formulation is provided as a suspension type. In several embodiments, formulations containing plant extracts will provide greater regulatory certainty if extracts are considered controlled substance and/or drugs.

### IV. Administration of Compositions

Certain embodiments of the disclosure relate to compositions and methods of administering the compositions.

The compositions, which may comprise the nanoparticles and active compounds of the disclosure, may be administered via a route of administration. In some embodiments, the composition is administered by more than one route of administration. In some embodiments, the composition is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

In some embodiments, the composition is administered at a dose of between 1mg/kg and 5000 mg/kg. In some embodiments, the composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 mg/kg.

The quantity to be administered, both according to number of treatments and dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the composition is one which can provide a sample level of the active compound at a concentration of about 1 μM to 150 μM. In another embodiment, the effective dose provides a sample level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following sample level of the agent that results from an active agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. The sample level may be analyzed from any biological sample, such as a blood sample, urine sample, skin sample, saliva sample, or the like. In certain embodiments, the active agent that is administered to a subject is metabolized in the body to a metabolized active agent, in which case the blood levels may refer to the amount of that agent. Alternatively, to the extent the active agent is not metabolized by a subject, the blood levels discussed herein may refer to the unmetabolized active agent.

Precise amounts of the active composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/mL or mM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at 1, 2, 3, 4, 5, 6, 7, 8, to 5, 6, 7, 8, 9, 10, 11, or 12 week intervals, including all ranges there between.

The compositions can be formulated for parenteral administration, e.g., formulated for injection via intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In certain embodiments, the form must be sterile and must be fluid to the extent that it may be easily injected. It also should be stable under the conditions of manufacture and storage and must be preserved against the contaminating action of microorganisms, such as bacteria and fungi.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

As disclosed elsewhere herein, some embodiments pertain to methods of treating a subject. In several embodiments, the method of treating comprises selecting patient for treatment. In several embodiments, the method of threating comprises administering to the patient an effective amount of a formulation comprising a nanoparticle composition comprising an active agent (e.g., pharmaceuticals, nutraceuticals, cosmetics, pigments, flavorings, and the like).

In several embodiments, compositions as described herein may be used to induce at least one effect, e.g. therapeutic effect, that may be associated with at least one active agent (e.g., pharmaceuticals, nutraceuticals, and the like), which is capable of inducing, enhancing, arresting or diminishing at least one effect, by way of treatment or prevention of unwanted conditions or diseases in a subject. As disclosed elsewhere herein, the at least one active agent may be selected amongst therapeutic agents, such as agents capable of inducing or modulating a therapeutic effect when administered in a therapeutically effective amount.

In several embodiments, the compositions disclosed herein can be used in methods of treatment and can be administered to a subject having a condition to be treated. In several embodiments, the subject is treated by administering an effective amount of a composition as disclosed herein to the subject.

In several embodiments, the disease or condition to be treated via administration of a composition as disclosed herein may include one or more of opioid withdrawal, pain relief, anxiety relief, depression, insomnia, inflammation, fever, fatigue, muscle aches, etc. In several embodiments, the nanoparticle composition (e.g., those including one or more active agents) is provided for use in treating a condition selected from pain associated disorders (as an analgesic), inflammatory disorders and conditions (as anti-inflammatory), appetite suppression or stimulation (as anoretic or stimulant), symptoms of vomiting and nausea (as antiemetic), intestine and bowl disorders, disorders and conditions associated with anxiety (as anxiolytic), disorders and conditions associated with psychosis (as antipsychotic), disorders and conditions associated with seizures and/or convulsions (as antiepileptic or antispasmodic), sleep disorders and conditions (as anti-insomniac), disorders and conditions which require treatment by immunosuppression, disorders and conditions associated with elevated blood glucose levels (as antidiabetic), disorders and conditions associated with nerve system degradation (as neuroprotectant), inflammatory skin disorders and conditions (such as psoriasis), disorders and conditions associated with artery blockage (as anti-ischemic), disorders and conditions associated with bacterial infections, disorders and conditions associated with fungal infections, proliferative disorders and conditions, disorders and conditions associated with inhibited bone growth, post trauma disorders, and others.

The active agent (substance, molecule, element, compound, entity, or a combination thereof) may be selected amongst therapeutic agents, such as agents capable of inducing or modulating a therapeutic effect when administered in a therapeutically effective amount, and non-therapeutic agents, such as agents which by themselves do not induce or modulate a therapeutic effect but which may endow the pharmaceutical composition with a selected desired characteristic.

In several embodiments, a nanoparticle compositions as disclosed herein may be selected to treat, prevent or ameliorate any pathology or condition. In several embodiments, administering of a therapeutic amount of the composition or system described herein, whether in a concentrate form or in a diluted formulation form, is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring or a combination of two or more of the above.

In several embodiments, as mentioned elsewhere herein, the compositions disclosed herein, may be provided in a number of different forms for administration and/or ingestion. In several embodiments, including when provided in a ready-to-drink beverage, the compositions are stable during ozonation sterilization, UV sterilization, heat sterilization, filtration sterilization, and/or gamma irradiation during beverage preparation and packaging. In several embodiments, the particle size and/or PDI after sterilization (e.g., exposure to techniques that allow sterilization of the composition) varies by less than or equal to about: 1%, 5%, 10%, 20%, 30%, or ranges including and/or spanning the aforementioned values. In several embodiments, the active agent (e.g., pharmaceuticals, nutraceuticals, cosmetics, pigments, flavorings, and the like) concentration after sterilization (e.g., exposure to techniques that allow sterilization of the composition) drops by less than or equal to about: 1%, 5%, 10%, 15%, or ranges including and/or spanning the aforementioned values. In several embodiments, including after stabilization, the beverages comprising nanoparticle compositions have a shelf life of equal to or greater than 6 months, 12 months, 14 months, 16 months, 18 months, 19months, 24 months, or ranges including and/or spanning the aforementioned values.

In several embodiments, the compositions are provided in a sterilized beverage. In several embodiments, the sterilized beverage may be a cold beverage (e.g., juices, sports drinks, energy drinks, protein drinks, nutritional drinks, sodas, etc.). In several embodiments, the cold beverage may be a carbonated beverage. In several embodiments, the cold beverage may be an alcoholic beverage. In several embodiments, the compositions may be provided in hot beverages (e.g., coffee, tea, etc.). In several embodiments, after a 30 minute period in a hot beverage, the particle size and/or PDI varies by less than or equal to about: 1%, 5%, 10%, 20%, 30%, or ranges including and/or spanning the aforementioned values. In several embodiments, after a 30 minute period in a hot beverage, the active agent (e.g., pharmaceuticals, nutraceuticals, cosmetics, pigments, flavorings, and the like) concentration drops by less than or equal to about: 1%, 5%, 10%, 15%, or ranges including and/or spanning the aforementioned values.

Several embodiments also encompass methods for administering the disclosed compositions. Multiple techniques of administering the nanoparticle compositions as disclosed herein exist including, but not limited to, oral, sublingual, buccal, rectal, topical, vaginal, acrosol, injection and parenteral delivery, including intramuscular, subcutaneous, intravenous, intramedullary injections, intrathecal, direct intraventricular, intraperitoneal, intranasal, and intraocular injections. In several embodiments, administration is performed through oral pathways, which administration includes administration in an emulsion, capsule, tablet, film, chewing gum, suppository, granule, pellet, spray, syrup, or other such forms. As further examples of such modes of administration and as further disclosure of modes of administration, disclosed herein are various methods for administration of the disclosed compositions including modes of administration through intraocular, intranasal, and intraauricular pathways. In several embodiments, oral formulations may comprise of DMSO and NMP.

In several embodiments, the nanoparticles (or compositions comprising them) may be used to deliver active compounds to a biomass. Thus, the biomass may be fortified with extracts, additional terpenes, and/or combinations thereof (as disclosed elsewhere herein). In several embodiments, the fortification is accomplished by spraying a liquid solution onto the biomass (or other consumer product). In several embodiments, by drying to completeness, a product that is fortified with an active is provided. In several embodiments, these fortifying therapeutic agents can be used to enhance health benefits of the consumer product (e.g., biomass), to change the flavor profile of the consumer product (e.g., biomass), to change the physiological effects of the consumer product (e.g., biomass), and/or to provide other benefits.

In several embodiments, where a topical is provided, the topical formulation may include SLM2026 (skin lipid matrix including Aqua (Water), Caprylic/Capric Triglyceride, Hydrogenated Phosphatidylcholine, Pentylene Glycol, Glycerin, Butyrospermum Parkii (Shea) Butter, Squalane, Ceramide NP), SLM2038 (skin lipid matrix including Aqua (and) Caprylic/Capric Triglyceride (and) Hydrogenated Phosphatidylcholine (and) Pentylene Glycol (and) Glycerin (and) Butyrospermum Parkii Butter (and) Squalane), or other formulated emulsion systems. In several embodiments, where a topical is provided, topical permeation enhancers may be included and may be selected from, but not inclusive of, the following: dimethyl sulfoxide, dimethyl sulfone, ethanol, propylene glycol, dimethyl isosorbide, polyvinyl alcohol, Capryol™ 90, Labrafil M1944 CS, Labrasol, Labrasol ALF, LauroglycolT M90, Transcutol HP, Capmul S12L, Campul PG-23 EP/NF, Campul PG-8 NF. The topical may include one or more of Lipoid's Skin Lipid Matrix 2026 technology, lipid/oil based ingredients or oil soluble ingredients, and includes Captex 170 EP as a skin permeation enhancer, argan oil, menthol, arnica oil, camphor, grapefruit seed oil, For example, dimethyl sulfoxide, dimethyl isosorbide, topical analgesics such as lidocaine, wintergreen oil, and terpenes such as guaiacol. In several embodiments, any one or more of these ingredients is present in the topical composition at a dry wt. % of equal to or less than about: 5%, 10%, 15%, 20%, 30%, 40%, 50%, 60%, or ranges including and/or spanning the aforementioned values. In several embodiments, any one or more of these ingredients is present in the topical at a wet wt. % of equal to or at least about: 2.5%, 5%, 7.5%, 10%, 12.5%, 15%, 20%, 30%, or ranges including and/or spanning the aforementioned values.

In several embodiments, the nanoparticle compositions disclosed herein can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. See, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). In several embodiments, these additional agents are not added. Such preparations can include liposomes, microemulsions, micelles, and/or unilamellar or multilamellar vesicles.

In several embodiments, the nanoparticle composition is configured for oral ingestion. In several embodiments, the nanoparticle formulation is provided as a drinkable solution, such as a beverage, elixir, tonic, or the like. In several embodiments, the nanoparticle formulation is provided as a powder that can be constituted in a liquid (e.g., water, juice, coffec) and ingested orally.

For administration (e.g., oral), the nanoparticle compositions can be provided as a tablet, capsule, pressed tablet, aqueous or oil suspension, dispersible powder or granule (as a food additive, drink additive, etc.), emulsion, hard or soft capsule, syrup or elixir. Compositions intended for oral use can include one or more of the following agents: sweeteners, flavoring agents, coloring agents and preservatives. In several embodiments, the compositions are provided in ready-to-drink formulations, such as protein drinks, energy drinks, sodas, juices, coffees, etc.

Formulations for oral use can also be provided as gelatin capsules. In several embodiments, a powder composition as disclosed herein is added to the gelatin capsule. In several embodiments, the active ingredient(s) in the nanoparticle compositions disclosed herein are mixed with an inert solid diluent, such as calcium carbonate, calcium phosphate, or kaolin, or as soft gelatin capsules. In soft capsules, the active compounds can be dissolved or suspended in suitable liquids, such as water. Stabilizers and microspheres formulated for oral administration can also be used. Capsules can include push-fit capsules made of gelatin, as well as soft, sealed capsules made of gelatin and a plasticizer, such as glycerol or sorbitol.

In capsule formulations, trehalose can be added. In several embodiments, trehalose is present in the nanoparticle composition at a dry wt. % of equal to or less than about: 5%, 10%, 15%, 20%, 30%, 40%, 50%, 60%, or ranges including and/or spanning the aforementioned values. In several embodiments, the trehalose is present in the composition at a wet wt. % of equal to or at least about: 2.5%, 5%, 7.5%, 10%, 12.5%, 15%, 20%, 30%, or ranges including and/or spanning the aforementioned values.

Dry powder formulations or liquid embodiments may also be used in a variety of consumer products. For example, in some embodiments, dry powders can be added (e.g., scooped, from a packet, squirted from a dispenser, etc.) into any consumer product (e.g., a hot or cold beverage).

In several embodiments, liquid solutions or powdered nanoparticle formulations can be coated onto and/or added into a consumer product (e.g., sprayed and/or squirted from a dispenser, through dipping, soaking, rolling, dusting, etc.). In several embodiments, the consumer product is a food product (e.g., candies, lollipops, edibles, food, ingestible, buccal adhesives, or others). In several embodiments, the consumer product is a biomass. In several embodiments, the nanoparticles (e.g., of the compositions disclosed herein) supplement and/or fortify the consumer product (e.g., biomass) with an active agent from the nanoparticles. In several embodiments, the active agent is delivered to the user in a greater quantity than would be achieved using (e.g., consuming) the biomass alone.

In several embodiments, the nanoparticle compositions may be used to improve a condition. In several embodiments, an improvement in a condition can be a reduction in disease symptoms or manifestations (e.g., opioid withdrawal symptoms, pain, anxiety & stress, mood disorders (e.g., depression), seizures, malaise, inflammation, insomnia, etc.). Actual dosage levels of active ingredients in an active composition of the presently disclosed subject matter can be varied so as to administer an amount of the active compound(s) that is effective to achieve the desired response for a particular subject and/or application. The selected dosage level will depend upon a variety of factors including, but not limited to, the activity of the composition, composition, route of administration, combination with other drugs or treatments, severity of the condition being treated, and the physical condition and prior medical history of the subject being treated. In several embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

In several embodiments, surprisingly, an aqueous nanoparticle composition comprising an active agent (e.g., pharmaceuticals, nutraceuticals, cosmetics, pigments, flavorings, and the like) as disclosed herein may be administered using an atomizer. In several embodiments, an atomizer nozzles are used in oral spray, such as the binaca spray. In several embodiments, an atomizer nozzle is used in a nasal spray. This result is surprising, as the extracts disclosed herein would be typically be understood to clog atomizer nozzles.

In several embodiments, as disclosed elsewhere herein, the nanoparticles or compositions may be used as coatings. In several embodiments, coating is performed with an aqueous or solvent solution of the nanoparticles. For example, the solution may be sprayed (e.g., via a spray nozzle, atomizer, etc.) or otherwise coated (e.g., dip-coated, etc.). In several embodiments, pharmaceutical coating equipment (e.g., that used to coat tablets, beads, drug layered/coated films) is used to coat the biomass. In several embodiments, fluid bed technology, film bed technology, dry powder laying technology, and/or combinations thereof are used to coat the biomass. In several embodiments, film coating is used.

In several embodiments, prior to coating with a liquid solution of nanoparticles, the material to be coated is dried completely. Then, after coating, the coated material may be dried. In other implementations, the material to be coated is not dried before being coated. In several embodiments, as disclosed elsewhere herein, a powder can be used to coat the material to be coated. In several embodiments, a powder nanoparticle formulation is dusted or coated onto the material to be coated. Additional drying may be performed to afford a consumable fortified product. In several embodiments, where the material to be coated is dried prior to coating with a powder nanoparticle, an additional drying step may optionally be performed (though it may not be required). In several embodiments, the dried fortified material is suitable for use by a user.

In several embodiments, the fortified material is further processed prior to use (e.g., in dried or undried form). In several embodiments, milling is used to reduce the size of the coated material particles. In several embodiments, the milling is a two stage process with a first course milling and then a fine milling. In some embodiment, after milling (dry or wet) the average particle size of the fortified material is such that greater than 50% pass through screen having a mesh size of less than or equal to 100, 150, 200, or ranges spanning and/or including the aforementioned values. In some embodiment, after milling (dry or wet) the average particle size of the fortified material is less than or equal to about: 1000 μm, 500 μm, 200 μm, or ranges including and/or spanning the aforementioned values. In several embodiments, after drying and/or milling, the fortified material is suitable for delivery to a user.

In several embodiments, the lipid particle formulation, can be remote loaded with active agents. In several embodiments, a liquid formulation of lipid particles is adding to an active agent. In several embodiments, the active agent incorporates into the particles by hydrophobic/hydrophilic interactions, electrostatic interactions, etc. In several embodiments, a remote loaded product could be coated onto biomass (as disclosed above), dried, and/or milled to provide a fortified, finished product. In several embodiments, the lipid particle can be provided with or without an active agent inside prior to remote loading. Then, other active agents can be loaded into that particle through remote loading.

In several embodiments, liquid formulations can be added measured and poured into any consumer product. In several embodiments, the consumer product can include one or more alcoholic beverages, milks (dairy, but also nuts “milks” such as almond juice, etc.), coffee, sodas, tea, fermented beverages, wines, nutritional supplements, smoothies, simple water, sports drinks, sparkling water, or the like. In several embodiments, the consumer product can include one or more eye drops, mouth wash, lotions/creams/serums, lip balms, hair care products, deodorant, nasal solutions, enema solutions, liquid soaps, solid soaps, or the like. In several embodiments, the consumer product can include one or more food products. In several embodiments, the consumer product can include desserts. In several embodiments, the consumer product can include single serving products of multi-serving products (e.g., family size). In several embodiments, the consumer product can include one or more dried products (e.g., flour, coffee creamer, protein shakes, nutritional supplements, etc.). In several embodiments, these dried products can be configured to be reconstituted for use. In several embodiments, the consumer product can include one or more the dried product can be added to other dietary supplements (e.g., multivitamins, gummies, etc.).

Several illustrative embodiments of compositions and methods have been disclosed. Although this disclosure has been described in terms of certain illustrative embodiments and uses, other embodiments and other uses, including embodiments and uses which do not provide all of the features and advantages set forth herein, are also within the scope of this disclosure. Components, elements, features, acts, or steps can be arranged or performed differently than described and components, elements, features, acts, or steps can be combined, merged, added, or left out in various embodiments. All possible combinations and subcombinations of elements and components described herein are intended to be included in this disclosure. No single feature or group of features is necessary or indispensable.

Certain features that are described in this disclosure in the context of separate implementations can also be implemented in combination in a single implementation. Conversely, various features that are described in the context of a single implementation also can be implemented in multiple implementations separately or in any suitable subcombination. Moreover, although features may be described above as acting in certain combinations, one or more features from a claimed combination can in some cases be excised from the combination, and the combination may be claimed as a subcombination or variation of a subcombination.

The agents in some aspects of the disclosure may be formulated into preparations for local delivery (such as to a specific location of the body, such as a specific tissue or cell type) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the disclosure also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain aspects, the pharmaceutical compositions are administered in the form of injectable compositions either as liquid solutions or suspensions; solid forms suitable or solution in, or suspension in, liquid prior to injection may also be prepared. These preparations also may be emulsified. In some embodiments, the composition comprises a pharmaceutically acceptable carrier. For instance, the composition may contain 10 mg or less, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyl oleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, antifungal agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. In further aspects, the pharmaceutical compositions may include classic pharmaceutical preparations.

Administration of compositions according to certain aspects may be via any common route so long as the target tissue is available via that route. This may include oral, nasal, buccal, rectal, vaginal or topical administration. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the acrosol may be between about 0.01 ml and 0.5 ml, for example.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular active substance. It is contemplated that other agents may be used in combination with certain aspects of the present embodiments to improve the therapeutic efficacy of treatment.

Any portion of any of the steps, processes, and/or compositions disclosed or illustrated in one embodiment, flowchart, or example in this disclosure can be combined or used with (or instead of) any other portion of any of the steps, processes, and/or compositions disclosed or illustrated in a different embodiment, flowchart, or example. The embodiments and examples described herein are not intended to be discrete and separate from each other. Combinations, variations, and other implementations of the disclosed features are within the scope of this disclosure.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### I. Example 1: Preparation of Embodiments of the Composition

Unless otherwise noted, the ingredients used herein were obtained from the following vendors: Sunflower derived phosphatidylcholine and medium chain triglycerides were purchased from American Lecithin Company (a Lipoid Company listed as “MCT”), potassium sorbate, peppermint oil, vitamin E, malic acid, and cholesterol were purchased from Spectrum Chemicals, cannabinoids were purchased from Botanical & Bioscience Laboratories, water for injection was purchased from Rocky Mountain Biologicals, and citric acid monohydrate and sodium benzoate were purchased from JT Baker, Sigma, Spectrum, or other commercial sources. The phosphatidylcholine was H 100-3 grade including over 96.3% or 99.9% phosphatidylcholine (hydrogenated). The phosphatidyl choline and less than 1.1% lysophosphatidylcholine and less than 2.0% triglycerides. This is a highly purity phosphatidylcholine (over 96% pure phosphatidylcholine (hydrogenated)). LIPOID® P LPC 80 is produced by American Lecithin Company. The product is 80% lysophosphatidylcholine from non-GMO soy lecithin and has enzyme modifications that enhance surfactant properties and water dispersibility. ALCOLEC® LEM is produced by American Lecithin Company. The food grade product is an amber fluid lecithin which has been enzyme modified to enhance water dispersibility and, in systems containing starch, to slow starch retrogradation. Effective oil-in-water emulsifier having an HLB of approximately 8. The product typically contains 14% phosphatidylcholine. VEGAPURE® 867 is produced by BASF and is a mixture of plant sterols and plant esters.

Particle size and zeta potential of liquid was measured on a Malvern ZS90 Zetasizer (Malvern, UK). Products were measured in low-volume, disposable cuvettes and zeta cassettes.

Active agent concentrations, related substances and identity (retention time) were measure by high-pressure liquid chromatography (HPLC) at 374 Labs (Reno, NV). Residual solvents and pesticides were measured by gas chromatography (GC), and heavy metals by inductive coupled plasma-optical emission spectrometry (ICP-OES) at 374 Labs. Rapid preservative effectiveness testing was determined by a reduction in colony forming units (CFU) of test microorganisms at Microchem Laboratory (Round Rock, Texas). Testing confirmed that the compositions were resistant to bacterial growth (by measuring colony forming units (CFUs) per volume in a given amount of time.

Manufacturing Process: Active agent containing-and non-active agent containing-lipid nanoparticles were prepared using a solvent-based method with high pressure homogenization. To prepare the nanoparticle composition lipophilic ingredients (hydrophobic active agents, medium chain triglyceride, cholesterol, phosphatidylcholine, Vitamin E, oil soluble flavoring, etc.) were accurately weighed onto a weigh boat and then transferred to a 20 liter, glass, round-bottom flask. To the lipophilic ingredients, approximately 1.3 to 1.5 times the weight of the lipophilic ingredients was added of 100% (200 proof) ethanol. The lipophilic ingredients were dissolved in the ethanol before proceeding. The 20 liter round bottom flask was transferred to a Hei-VAP Industrial Rotary Evaporator (Heidolph Corporation) and the ethanol was removed by evaporation under reduced pressure, elevated temperature, and vessel rotation. When the ethanol was removed, a film of lipid remained on the glass vessel walls. The lipid film was blanketed with nitrogen glass and left at room temperature overnight.

All water-soluble formulation ingredients (water soluble flavoring, water soluble active agents, sodium benzoate, potassium sorbate, citric acid monohydrate, malic acid, etc.) were dissolved into water for injection at the specified concentrations. Aqueous solutions were heated and filtered prior to further use. An appropriate amount of aqueous solution was transferred to the glass vessel containing the dried lipid ingredients. The glass vessel was transferred to a heating mantel and warmed with constant stirring from an overhead mixer. Mixing was continued until a homogenous slurry of lipids in water was formed. The full volume of lipid slurry was processed through a microfluidizer (Microfluidics Corporation) 0to 10 times at a processing pressure of 10,000-30,000 PSI. Alternatively, the volume of lipid slurry can be processed at a pressure of 10,000-30,000 PSI such that the material is recirculated back into the unprocessed volume for a period of time until the desired particle size characteristics are achieved. The resulting lipid nanoparticle solution was cooled with continuous stirring for 12-24 hours before characterizing and fill-finish. Flavoring in oil form was introduced into the dried lipid film prior to introduction of the aqueous solution. Water soluble flavoring is dissolved into the water for injection prior to introduction into the lipid film.

### II. Example 2: Preparation of Nanoparticles and Stability Study

Multiple nanoparticles were prepared using the following methods.

Manufacturing Process for active agent-and non-active agent-containing formulations except Ashwagandha extract containing formulations: Active agent containing-and non-active agent containing-lipid nanoparticles were prepared using a solvent-free method with high pressure homogenization. To prepare the nanoparticle composition, all water-soluble formulation ingredients (sodium benzoate, potassium sorbate, citric acid monohydrate, etc.) were dissolved into water for injection at the specified concentrations. Aqueous solutions were heated to formulation temperature. High shear overhead mixing was applied. Lipophilic ingredients (hydrophobic active agents, medium chain triglyceride, sterols, phosphatidylcholine, Vitamin E, etc.) were accurately weighed onto a weigh boat and then transferred to a glass beaker with continued high shear mixing. Mixing was continued until a homogenous slurry of lipids in water was formed. The full volume of lipid slurry was processed through a microfluidizer (Microfluidics Corporation) 0 to 10 times at a processing pressure of 10,000-30,000 PSI. Alternatively, the volume of lipid slurry can be processed at a pressure of 10,000-30,000 PSI such that the material is recirculated back into the unprocessed volume for a period of time until the desired particle size characteristics are achieved. The resulting lipid nanoparticle solution was cooled for 12-24 hours before characterizing and fill-finish.

Manufacturing Process for Ashwagandha extract containing formulation: Active agent Ashwagandha extract containing-lipid nanoparticles were prepared using a solvent-based method with high pressure homogenization. Ashwagandha extract was first combined with 100% (200 proof) ethanol. The mass of ethanol used was equal to 1.3 to 1.5 times the combined mass of the other lipophilic ingredients (medium chain triglyceride, sterols, phosphatidylcholine, Vitamin E, etc.) used. The mixture was stirred until ashwagandha actives had been dissolved in the ethanol. Ethanol insolubles were removed by filtration. The remaining nanoparticle composition lipophilic ingredients (medium chain triglyceride, sterols, phosphatidylcholine, Vitamin E, etc.) were accurately weighed onto a weigh boat and then transferred to a 20 liter, glass, round-bottom flask. To the lipophilic ingredients, the ethanol used to dissolve the ashwagandha actives was added. The other lipophilic ingredients were dissolved in the ethanol solution before proceeding. The 20 liter round bottom flask was transferred to a Hei-VAP Industrial Rotary Evaporator (Heidolph Corporation) and the ethanol was removed by evaporation under reduced pressure, elevated temperature, and vessel rotation. When the ethanol was removed, a film of lipid remained on the glass vessel walls. The lipid film was blanketed with nitrogen glass and left at room temperature overnight.

All water-soluble formulation ingredients (sodium benzoate, potassium sorbate, citric acid monohydrate, etc.) were dissolved into water for injection at the specified concentrations. Aqueous solutions were heated and filtered prior to further use. An appropriate amount of aqueous solution was transferred to the glass vessel containing the dried lipid ingredients. The glass vessel was transferred to a heating mantel and warmed with constant stirring from an overhead mixer. Mixing was continued until a homogenous slurry of lipids in water was formed. The full volume of lipid slurry was processed through a microfluidizer (Microfluidics Corporation) 0 to 10 times at a processing pressure of 10,000-30,000 PSI. Alternatively, the volume of lipid slurry can be processed at a pressure of 10,000-30,000 PSI such that the material is recirculated back into the unprocessed volume for a period of time until the desired particle size characteristics are achieved. The resulting lipid nanoparticle solution was cooled with continuous stirring for 12-24 hours before characterizing and fill-finish.

Testing of the formulations were performed. 250 gram batches containing the concentrations of components described in Table 1 below were prepared and 30 grams were filled into 30 mL dropper cap bottles for stability testing. Samples were stored at controlled 2-8° C., room temperature (25° C./60% relative humidity), or accelerated conditions (40° C./75% relative humidity) for testing. Shown in Table 2, Table 3, and Table 4 are changes in concentration of the active agent, changes in Z-average particle size, and changes in Polydispersity Index (PDI) over the period of time tested for each storage condition.

The formulations show stability over months of storage under the respective conditions. As an example, when the coenzyme Q10 containing nanoparticles were stored at controlled room temperature for 3 months, the coenzyme Q10 concentration changed by +0.3% (essentially unchanged), respectively. The Z-average particle size changed from 73.06 nm to 70.77 nm (essentially unchanged) during this time, while the PDI decreased from 0.128 to 0.104 (essentially unchanged).

When the coenzyme Q10 containing nanoparticles were stored at accelerated temperature for 3 months, the coenzyme Q10 concentration changed by −7.9%, respectively. The Z-average particle size increased from 73.06 nm to 89.21 nm during this time, while the PDI increased from 0.128 to 0.252.

Storage at 40° C./75% is generally understood to increase degradation kinetics by a factor of 4 compared to controlled room temperature based on the Arrhenius equation. A loss of only 7.9% potency over 3 months stored under these conditions suggests that the concentration will remain above 90% of the formulated target for longer than 3 months, but likely less than 6 months. Therefore, coenzyme Q10 stability is expected to be between 1 and 2 years of storage at controlled room temperature. This agrees well with observed stability at 25° C./60% relative humidity showing no significant signs of degradation after 3 months.

As another example, when the beta carotene containing nanoparticles were stored at controlled room temperature for 3 months the beta carotene concentration changed by −3.1% (essentially unchanged), respectively. The Z-average particle size changed from 271.3 nm to 400.4 nm during this time, while the PDI decreased from 0.806 to 0.415.

When the beta carotene containing nanoparticles were stored at accelerated temperature for 3 months the beta carotene concentration changed by −11.7%, respectively. The Z-average particle size increased from 271.3 nm to 583.3 nm during this time, while the PDI decreased from 0.806 to 0.407.

Storage at 40° C./75% is generally understood to increase degradation kinetics by a factor of 4 compared to controlled room temperature based on the Arrhenius equation. A loss of 11.7% potency over 3 months stored under these conditions suggests that the concentration will remain above 90% of the formulated target for 2 to 3 months. Therefore, beta carotene stability is expected to be between 8 months and 1 year of storage at controlled room temperature. This agrees well with observed stability at 25° C./60% relative humidity showing 3.1% degradation after 3 months.

TABLE 1

| | Percent of ingredients | | | | | | | | |
|---|---|---|---|---|---|---|---|---|---|
| | Active ingredient | | | VEGAPURE ® | DL alpha | Potassium | Sodium | Citric | |
| Formulation | and concentration | HSPC | MCT | 867 | Tocopherol | Sorbate | Benzoate | Acid | Water |
| Formulation 1 - | Vitamin K2 | 10 | 9.3 | 1 | 0.1 | 0.1 | 0.1 | 0.1 | 79.29 |
| Vitamin K2 | (powder) 0.01 | | | | | | | | |
| Formulation 2 - | Vitamin K2 | 10 | 9.3 | 1 | 0.1 | 0.1 | 0.1 | 0.1 | 79.29 |
| Vitamin K2 | (oil) 0.002 | | | | | | | | |
| Formulation 3 - | Additional plant | 15.0 | 13.5 | 1.5 | 0.1 | 0.1 | 0.1 | 0.1 | 69.6 |
| Plant Sterol | sterol in the form | | | | | | | | |
| | of VEGAPURE ® | | | | | | | | |
| | 867 and HSPC | | | | | | | | |
| Formulation 4 - | Co-Q10 1.0 | 10 | 9.3 | 1.0 | 0.1 | 0.1 | 0.1 | 0.1 | 78.3 |
| Co-Q10 | | | | | | | | | |
| Formulation 5 | Replacement of | 13 | 13.5 | 1.5 | 0.1 | 0.1 | 0.1 | 0.1 | 69.6 |
| Lysophosphatidylcholine | HSPC with | | | | | | | | |
| | LIPOID ® P LPC | | | | | | | | |
| | 80 at 2.0 | | | | | | | | |
| Formulation 6 - | Replacement of | — | 13.5 | 1.5 | 0.1 | 0.1 | 0.1 | 0.1 | 69.6 |
| Lysophospholipids | HSPC with | | | | | | | | |
| | ALCOLEC ® | | | | | | | | |
| | LEM at 15.0 | | | | | | | | |
| Formulation 7 - | Phytoceramides | 11.636 | 12.488 | 1.5 | 0.1 | 0.1 | 0.1 | 0.1 | 69.6 |
| Phytoceramides | (oil) 4.36 | | | | | | | | |
| Formulation 8 - | DHA 2.0 | 10 | 7.5 | 1 | 0.1 | 0.1 | 0.1 | 0.1 | 79.1 |
| Docosahexaenoic | | | | | | | | | |
| Acid | | | | | | | | | |
| Formulation 9 - | Ashwagandha | 10 | 4.5 | 1 | 0.1 | 0.1 | 0.1 | 0.1 | 78.3 |
| Ashwagandha | Extract 1.0 | | | | | | | | |
| Formulation 10 - | Curcumin 0.517 | 10 | 8.78 | 1 | 0.1 | 0.1 | 0.1 | 0.1 | 79.3 |
| Curcumin | | | | | | | | | |
| Formulation 11 - | Beta Carotene 1.0 | 10 | 9.3 | 1 | 0.1 | 0.1 | 0.1 | 0.1 | 78.3 |
| Beta Carotene | | | | | | | | | |
| Formulation 12 - | Squalene Oil 5.0 | 10 | 4.5 | 1 | 0.1 | 0.1 | 0.1 | 0.1 | 79.1 |
| Squalene Oil | | | | | | | | | |
| Formulation 13 - | Additional d/l- | 10 | 4.5 | 1 | 5.0 | 0.1 | 0.1 | 0.1 | 79.2 |
| Vitamin E | alpha tocopherol | | | | | | | | |
| Formulation 14 - | Vitamin D3 0.1 | 10 | 9.3 | 1 | 0.1 | 0.1 | 0.1 | — | 79.3 |
| Vitamin D3 | | | | | | | | | |

TABLE 2

| | | Potency (mg/g) | | | | | | | |
|---|---|---|---|---|---|---|---|---|---|
| | Temp | | 1 | 2 | 3 | 4 | 6 | 9 | 12 |
| Sample | C. | Initial | month | month | month | month | month | month | month |
| Lysophosphatidylcholine | 25 | 15.9 | 19.23 | 23.25 | 28.17 | — | 34.15 | 38.55 | 43.72 |
| | 40 | — | 24.83 | 34.56 | 42.76 | — | 43.78 | — | — |
| | 4 | — | — | — | — | — | — | — | — |
| Plant Sterols | 25 | 13.4 | 12.76 | 13 | 13.2 | — | 13.7 | 14.1 | 13.6 |
| | 40 | — | 12.51 | 13 | 13.4 | — | 13.3 | — | — |
| | 4 | — | 12.26 | — | — | — | — | — | — |
| Co-Q10 | 25 | 8.8176 | 9.3 | 5.42 | 9.1 | — | 9.513 | 8.531 | 11.418 |
| | 40 | — | 8.9 | 5.07 | 8.21 | — | 7.321 | — | — |
| | 4 | — | — | — | — | — | — | — | — |
| Vitamin E | 25 | 34.6 | 27.261 | 44.09 | 37.909 | — | 32.064 | 160.807 | 42.076 |
| | 40 | — | 27.494 | 38.91 | 32.565 | — | 30.561 | — | — |
| | 4 | — | 28.193 | — | — | — | — | — | — |
| Beta Carotene | 25 | 2.7555 | 3.18 | 1.55 | 2.67 | — | 2.794 | 2.367 | |
| | 40 | — | 2.62 | 1.12 | 2.16 | — | 2.052 | — | |
| | 4 | — | — | — | — | — | — | — | |
| Squalene Oil | 25 | 0.0033 | 47.1088 | 45.6843 | 43.89443 | — | 60.683 | | |
| | 40 | — | 45.2051 | 45.8994 | 46.52148 | — | 56.512 | | |
| | 4 | — | 47.138 | — | — | — | — | | |
| Vitamin K | 25 | 0.0666 | 0.0433 | 0.0433 | 0.1 | — | 0.051 | | |
| (Powder) | 40 | — | 0.0433 | 0.0433 | 0.1 | — | — | | |
| | 4 | — | — | — | — | — | — | | |
| DHA | 25 | 19.98 | 17.32 | 17.32 | 16.65 | — | 16.65 | 16.65 | 16.65 |
| | 40 | — | 17.32 | 17.32 | 16.65 | — | 13.32 | — | — |
| | 4 | — | — | — | — | — | — | — | — |
| curcumin | 25 | 0.333 | 8.66 | 0.433 | 0.21978 | — | 0.17982 | 0.20313 | 0.1998 |
| | 40 | — | 9.562 | 0.433 | 0.20313 | — | — | — | — |
| | 4 | — | — | — | — | — | — | — | — |
| phytoceramides | 25 | | | | | | | | |
| | 40 | | | | | | | | |
| | 4 | | | | | | | | |
| Vitamin K (Oil) | 25 | 0 | 0.0433 | 0.0666 | 0.045534 | — | 0.051 | 0.05 | |
| | 40 | — | 0.0433 | 0.0333 | 0.049659 | — | 0.066 | — | |
| | 4 | — | — | — | — | — | — | — | |
| Ashwagandha | 25 | 0.999 | 0.113 | 0.0529 | — | — | — | 0.0666 | 0 |
| | 40 | — | 0.111 | 0.0473 | — | — | — | — | — |
| | 4 | — | — | — | — | — | — | — | — |
| Lysophospholipids | 25 | 15.9 | 19.23 | 23.25 | 28.17 | — | 34.15 | 38.55 | 43.72 |
| | 40 | — | 24.83 | 34.56 | 42.76 | — | 43.78 | — | — |
| | 4 | — | — | — | — | — | — | — | — |
| Vitamin D3 | 25 | 0.916 | — | — | — | 0.72 | 0.872 | 0.643 | 0.189 |
| | 40 | — | — | — | — | — | 0.728 | — | — |
| | 4 | — | — | — | — | — | — | — | — |

TABLE 3

| | | Particle Size (z-avg, nm) | | | | | | | |
|---|---|---|---|---|---|---|---|---|---|
| | Temp | | 1 | 2 | 3 | 4 | 6 | 9 | 12 |
| Sample | C. | Initial | month | month | month | month | month | month | month |
| Lysophosphatidylcholine | 25 | 107.97 | 148.4 | 152.93 | 158.77 | — | 156.13 | 127.8 | 123.2667 |
| | 40 | — | 161.1 | 172.53 | 126.07 | — | 182.6 | — | — |
| | 4 | — | 145.1 | 198.6 | 239.47 | — | 201.47 | 308.633 | 241.9 |
| Plant Sterols | 25 | 69.423 | 69.12 | 69.61 | 69.43 | — | 67.88 | 85.0267 | 71.21 |
| | 40 | — | 69.4 | 73.95 | 100.84 | — | 557.43 | — | — |
| | 4 | — | 96.51 | 97.42 | 103.62 | — | 96.31 | 85.267 | 105.6 |
| Co-Q10 | 25 | 73.06 | 70.69 | 71.04 | 70.77 | — | 69.53 | 100.59 | 76.7433 |
| | 40 | — | 70.68 | 81.42 | 89.21 | — | 595.8 | — | — |
| | 4 | — | 104.1 | 107.1 | 107 | — | 110.27 | 114.267 | 111.2 |
| Vitamin E | 25 | 114.9 | 124.4 | 119.13 | 126.93 | — | 123.87 | 119.233 | 130.6 |
| | 40 | — | 157 | 208.43 | 165.73 | — | 204.23 | — | — |
| | 4 | — | 119.6 | 115.57 | 121.87 | — | 122.07 | 120.533 | 120.567 |
| Beta Carotene | 25 | 271.33 | 299.233 | 82.93 | 400.43 | — | 418.53 | 496.53 | 776.333 |
| | 40 | — | 381.033 | 93.3 | 583.3 | — | 1230.5 | — | — |
| | 4 | — | 260.033 | 104.7 | 387.5 | — | 347.27 | 775.33 | 909.933 |
| Squalene Oil | 25 | 79.02 | 77.1733 | 71.69 | 82.96 | — | 73.63 | 143.03 | 96.5 |
| | 40 | — | 81.1133 | 75.01 | 119.87 | — | 471.47 | — | — |
| | 4 | — | 105.267 | 104.33 | 109.97 | — | 131 | 117.567 | 116 |
| Vitamin K (Powder) | 25 | 867.87 | 809.2 | 5685.33 | 658.07 | — | 142.1 | 141.9 | 134.8 |
| | 40 | — | 652.3 | 144.37 | 147.83 | — | 674.57 | — | — |
| | 4 | — | 1481 | 3221.33 | 876.1 | — | 3924.67 | 867.867 | 1201.8 |
| DHA | 25 | 167.33 | 152.5 | 151.73 | 180.83 | — | 226.1 | 186.5 | 193.167 |
| | 40 | — | 181 | 167.57 | 162.27 | — | 641.8 | — | — |
| | 4 | — | 160.9 | 149.73 | 168.3 | — | 172.27 | 138.53 | 163.367 |
| curcumin | 25 | 83.576 | 97.33 | 105.73 | 107.93 | — | 101.7 | 126.9 | 135.1 |
| | 40 | — | 99.94 | 128.77 | 126.13 | — | 223.47 | | — |
| | 4 | — | 123 | 123.93 | 125.87 | — | 123.07 | 135.83 | 142.167 |
| phytoceramides | 25 | 180.97 | 196.13 | 203.7 | 203.83 | — | 193.3 | 255.367 | 289.033 |
| | 40 | — | 213.67 | 201.83 | 198.93 | — | 354.9 | | — |
| | 4 | — | 209.5 | 210.7 | 209.8 | — | 216.6 | 214.433 | 221.233 |
| Vitamin K (Oil) | 25 | 84.36 | 81.85 | 78.45 | 81.03 | — | 82.92 | 92.043 | 92.52 |
| | 40 | — | 104.81 | 131.77 | 148.5 | — | 253.3 | — | — |
| | 4 | — | 118.5 | 122.97 | 122.2 | — | 119.7 | 153.8 | 137.9 |
| Ashwagandha | 25 | 70.07 | 94.22 | 79.36 | 83.8 | — | 77.26 | 103.6 | 103.5 |
| | 40 | — | 110.4 | 131.97 | 144.3 | — | 246 | — | — |
| | 4 | — | 97.75 | 99.08 | 101.93 | — | 107.9 | 116.067 | 127.933 |
| Lysophospholipids | 25 | 134.93 | 142.4 | 140.4 | 140.5 | — | 144.5 | 147.6 | 151.53 |
| | 40 | — | 146.63 | 154.6 | 159.2 | — | 232.4 | — | — |
| | 4 | — | 136.57 | 138.17 | 135.5 | — | 139.1 | 140.767 | 140.13 |
| Vitamin D3 | 25 | 67.88 | — | 71.7 | 67.37 | — | 67.42 | 131.9 | 68.98 |
| | 40 | — | — | 72.1 | 68.32 | — | 66.3 | — | — |
| | 4 | — | — | — | — | — | — | — | — |

TABLE 4

| | | PDI | | | | | | | |
|---|---|---|---|---|---|---|---|---|---|
| | Temp | | 1 | 2 | 3 | 4 | 6 | 9 | 12 |
| Sample | C. | Initial | month | month | month | month | month | month | month |
| Lysophosphatidylcholine | 25 | 0.35533 | 0.494 | 0.696 | 0.8 | — | 0.766 | 0.4913 | 0.44667 |
| | 40 | — | 0.552 | 0.799 | 0.523 | — | 0.569 | — | — |
| | 4 | — | 0.633 | 0.533 | 0.721 | — | 0.775 | 0.7263 | 0.69633 |
| Plant Sterols | 25 | 0.06433 | 0.073 | 0.065 | 0.066 | — | 0.053 | 0.14867 | 0.106 |
| | 40 | — | 0.084 | 0.209 | 0.227 | — | 0.454 | — | — |
| | 4 | — | 0.102 | 0.129 | 0.113 | — | 0.13 | 0.14567 | 0.1613 |
| Co-Q10 | 25 | 0.128 | 0.086 | 0.075 | 0.104 | — | 0.102 | 0.228 | 0.116 |
| | 40 | — | 0.096 | 0.311 | 0.252 | — | 0.314 | — | — |
| | 4 | — | 0.139 | 0.139 | 0.137 | — | 0.135 | 0.213 | 0.191 |
| Vitamin E | 25 | 0.112 | 0.115 | 0.133 | 0.216 | — | 0.117 | 0.1047 | 0.12867 |
| | 40 | — | 0.103 | 0.539 | 0.101 | — | 0.087 | — | — |
| | 4 | — | 0.101 | 0.101 | 0.082 | — | 0.072 | 0.113 | 0.092 |
| Beta Carotene | 25 | 0.806 | 0.47267 | 0.222 | 0.415 | — | 0.316 | 0.2046 | 0.383 |
| | 40 | — | 0.81667 | 0.254 | 0.261 | — | 0.748 | — | — |
| | 4 | — | 0.41567 | 0.116 | 0.407 | — | 0.361 | 0.32267 | 0.404 |
| Squalene Oil | 25 | 0.123 | 0.13 | 0.107 | 0.196 | — | 0.121 | 0.2746 | 0.24433 |
| | 40 | — | 0.21433 | 0.304 | 0.43 | — | 0.435 | — | — |
| | 4 | — | 0.142 | 0.145 | 0.141 | — | 0.2 | 0.23233 | 0.21733 |
| Vitamin K (Powder) | 25 | 1 | 0.955 | 0.6 | 0.91 | — | 0.17 | 0.27033 | 0.2323 |
| | 40 | — | 1 | 0.336 | 0.261 | — | 0.409 | — | — |
| | 4 | — | 1 | 0.562 | 0.339 | — | 0.589 | 0.62367 | 0.6203 |
| DHA | 25 | 0.1143 | 0.075 | 0.148 | 0.311 | — | 0.363 | 0.07067 | 0.085 |
| | 40 | — | 0.197 | 0.121 | 0.087 | — | 0.474 | — | — |
| | 4 | — | 0.126 | 0.123 | 0.168 | — | 0.244 | 0.13 | 0.186 |
| curcumin | 25 | 0.11 | 0.128 | 0.24 | 0.3 | — | 0.232 | 0.2333 | 0.22367 |
| | 40 | — | 0.125 | 0.213 | 0.076 | — | 0.216 | — | — |
| | 4 | — | 0.2 | 0.197 | 0.21 | — | 0.21 | 0.2983 | 0.31533 |
| phytoceramides | 25 | 0.10167 | 0.075 | 0.045 | 0.08 | — | 0.09 | 0.21 | 0.16967 |
| | 40 | — | 0.058 | 0.031 | 0.07 | — | 0.352 | — | — |
| | 4 | — | 0.083 | 0.088 | 0.083 | — | 0.094 | 0.066 | 0.035 |
| Vitamin K (Oil) | 25 | 0.214 | 0.206 | 0.225 | 0.212 | — | 0.22 | 0.213 | 0.2263 |
| | 40 | — | 0.284 | 0.402 | 0.218 | — | 0.344 | — | — |
| | 4 | — | 0.198 | 0.2 | 0.2 | — | 0.173 | 0.36967 | 0.207 |
| Ashwagandha | 25 | 0.148 | 0.201 | 0.21 | 0.297 | — | 0.193 | 0.2 | 0.20367 |
| | 40 | — | 0.097 | 0.075 | 0.137 | — | 0.217 | — | — |
| | 4 | — | 0.119 | 0.134 | 0.136 | — | 0.148 | 0.2233 | 0.13433 |
| Lysophospholipids | 25 | 0.1443 | 0.161 | 0.122 | 0.127 | — | 0.114 | 0.107 | 0.085 |
| | 40 | — | 0.105 | 0.069 | 0.074 | — | 0.039 | — | — |
| | 4 | — | 0.155 | 0.142 | 0.156 | — | 0.132 | 0.146 | 0.13967 |
| Vitamin D3 | 25 | 0.068 | — | 0.098 | 0.056 | — | 0.096 | 0.203 | 0.046 |
| | 40 | — | — | 0.122 | 0.128 | — | 0.086 | — | — |
| | 4 | — | — | — | — | — | — | — | — |

### III. Example 3: Preparation of Nanoparticles with Xanthan Gum and Stability Study in Hot Liquids and Over Time

Nanoparticles of Table 1 were prepared according to the method disclosed in Example 1, but 0.1 wt. % of water was replaced with xanthan gum for a total concentration of xanthan gum of 0.1 wt. %.

Stability of the xanthan gum containing nanoparticles in a hot liquid was tested. It was found by visual inspection that inclusion of xanthan gum in the nanoparticle improved stability of the nanoparticles in the hot liquid. Coffee was used as the hot liquid, with a temperature of 85° C.

Further, samples were stored at controlled 2-8° C., room temperature (25° C./60% relative humidity), or accelerated conditions (40° C./75% relative humidity) for testing. Shown in Table 5 are changes in concentration of the active agent, changes in Z-average particle size, and changes in Polydispersity Index (PDI) over the period of time tested for each storage condition.

TABLE 5

| | | Potency (mg/g) | | | | Particle size (z-avg, nm) | | | | PDI | | | |
|---|---|---|---|---|---|---|---|---|---|---|---|---|---|
| | Temp | | 1 | 2 | 3 | | 1 | 2 | 3 | | 1 | 2 | 3 |
| Sample | C. | Initial | month | months | months | Initial | month | months | months | Initial | month | months | months |
| Plant Sterols + 0.1 | 25 | 13.5 | | | | 217.8 | | | | 0.464 | | | |
| wt. % xanthan gum | 40 | | | | | | | | | | | | |
| | 4 | | | | | | | | | | | | |
| Co-Q10 + 0.1 wt. | 25 | 9.49 | | | | 224.03 | | | | 0.284 | | | |
| % xanthan gum | 40 | | | | | | | | | | | | |
| | 4 | | | | | | | | | | | | |
| Beta Carotene + 0.1 | 25 | 1.51 | | | | 201.8 | | | | 0.791 | | | |
| wt. % xanthan gum | 40 | | | | | | | | | | | | |
| | 4 | | | | | | | | | | | | |

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and 10 modifications apparent to those skilled in the art are deemed to be within the spirit, scope and concept of the disclosure as defined by the appended claims.

What is claimed is:

1. A nanoparticle composition comprising water at about 60 wt. % to about wt. 93.799% and at least one nanoparticle, the nanoparticle comprising:
at least one active agent at 0.001 wt. % to 20 wt. %;
at least one phospholipid at 5 wt. % to 20 wt. %;
at least one triglyceride at 1 wt. % to 15 wt. %;
at least one sterol at 0.2 wt. % to 2 wt. %; and
optionally at least one surfactant at 0 wt. % to 17.5 wt. %,
wherein the weight percentages are based on the weight of the composition.

2. The nanoparticle composition of claim 1, wherein the active agent comprises a hydrophobic active ingredient.

3. The nanoparticle composition of claim 1, wherein the active agent comprises coenzyme Q10, a vitamin E, a beta carotene, squalene, a vitamin K, a docosahexaenoic acid, a curcuminoid, a phytoceramide, vitamin D3, and/or an ashwagandha extract.

4. The nanoparticle composition of claim 1, wherein the active agent comprises a small molecule, wherein the triglyceride comprises a medium chain triglyceride, and wherein the phospholipid comprises a lysophospholipid, a phosphatidylcholine, and/or a phosphatidylserine.

5. The nanoparticle composition of claim 1, comprising the surfactant and the surfactant comprises an emulsifier.

6. The nanoparticle composition of claim 1, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.

7. The nanoparticle composition of claim 1, further comprising at least one co-emulsifier and/or at least one preservative.

8. The nanoparticle composition of claim 7, wherein the co-emulsifier is a gum.

9. The nanoparticle composition of claim 1, wherein the at least one nanoparticle comprises:
at least one triglyceride comprising capric and caprylic triglycerides;
DL alpha tocopherol;
a plant sterol and/or cholesterol;
optionally xanthan gum; and
optionally hydrogenated phosphatidylcholine.

10. A nanoparticle composition comprising water at about 60 wt. % to about wt. 93.799% and at least one nanoparticle, the nanoparticle comprising:
at least one phospholipid at 5 wt. % to 20 wt. %;
at least one triglyceride at 1 wt. % to 15 wt. %;
at least one sterol at 0.2 wt. % to 2 wt. %; and
optionally at least one surfactant at 0 wt. % to 17.5 wt. %,
wherein the weight percentages are based on the weight of the composition.

11. The nanoparticle composition of claim 10, wherein the triglyceride comprises a medium chain triglyceride and the phospholipid comprises a lysophospholipid, a phosphatidylcholine, and/or a phosphatidylserine.

12. The nanoparticle composition of claim 10, comprising the surfactant and the surfactant comprises an emulsifier.

13. The nanoparticle composition of claim 10, wherein the composition comprises a mixture of nanoparticles selected from at least two of a multilamellar nanoparticle vesicles, unilamellar nanoparticle vesicles, multivesicular nanoparticles, emulsion particles, irregular particles with lamellar structures and bridges, partial emulsion particles, combined lamellar and emulsion particles, micelles, and/or combinations thereof.

14. The nanoparticle composition of claim 10, further comprising at least one co-emulsifier and/or at least one preservative.

15. The nanoparticle composition of claim 14, wherein the co-emulsifier is a gum and the composition comprises 0.01 wt. % to 5 wt. % of the gum, wherein the weight percentages are based on the weight of the composition.

16. The nanoparticle composition of claim 15, wherein the at least one nanoparticle comprises:
at least one triglyceride comprising capric and caprylic triglycerides;
DL alpha tocopherol;
a plant sterol and/or cholesterol;
optionally xanthan gum; and
optionally hydrogenated phosphatidylcholine,
wherein the weight percentages are based on the weight of the composition.

17. A method of manufacturing a nanoparticle composition of claim 1, the method comprising the steps of:
(a) adding one or more phospholipid, one or more triglyceride, one or more sterol, optionally one or more active agents, and optionally one or more surfactants to water;
(b) mixing the ingredients of step (a) creating a mixture;
(c) homogenizing the mixture creating a homogenized mixture;
(d) performing microfluidization on the homogenized mixture creating a microfluid;
(e) sonicating the microfluid creating a sonicated microfluid;
(f) stirring the sonicated microfluid creating a stirred microfluid;
(g) creating a coacervation from the stirred microfluid; and
(h) precipitating the coacervation.

18. A nanoparticle composition comprising water at about 0 wt. % to about wt. 10% and at least one nanoparticle, the nanoparticle comprising:
at least one phospholipid at 10 wt. % to 50 wt. %;
at least one triglyceride at 2 wt. % to 37.5 wt. %;
at least one sterol at 0.4 wt. % to 5 wt. %;
optionally at least one active agent at 0.002 wt. % to 50 wt. %;
optionally at least one gum at 0.01 wt. % to 5 wt. %; and
optionally at least one surfactant at 0 wt. % to 43.75 wt. %.

19. The nanoparticle composition of claim 18, wherein the composition comprises a mixture of nanoparticles selected from at least two of a liposome, a micelle, a nanoemulsion, a multi-lamellar particle, a double liposome particle, and a solid lipid particle.

20. The nanoparticle composition of claim 19, wherein the at least one nanoparticle comprises:
at least one triglyceride comprising capric and caprylic triglycerides;
DL alpha tocopherol;
a plant sterol and/or cholesterol;
optionally xanthan gum; and
optionally hydrogenated phosphatidylcholine.
